(19) United States
(12) Patent Application Publication
(10) Pub. No.: US 2018/0172712 A1
(43) Pub. Date: Jun. 21, 2018

(54) METHODS AND MATERIALS FOR ASSESSING HYDROGEN PEROXIDE ACCUMULATION IN CELLS

(71) Applicant: Mayo Foundation for Medical Education and Research, Rochester, MN (US)

(72) Inventors: Sinto Sebastian Chirackal, Scottsdale, AZ (US); Rafael Fonseca, Scottsdale, AZ (US)

(73) Assignee: Mayo Foundation for Medical Education and Research, Rochester, MN (US)

(21) Appl. No.: 15/737,219

(22) PCT Filed: Jun. 16, 2016
(86) PCT No.: PCT/US16/37899

§ 371 Date: Dec. 15, 2017

Related U.S. Application Data
(60) Provisional application No. 62/180,327, filed on Jun. 16, 2015.

(51) Int. Cl. G01N 33/84; G01N 33/574; C12M 1/34; G01N 33/50; G01N 21/76; G01N 21/77

(52) U.S. Cl. CPC G01N 33/84; G01N 33/574; C12M 41/38; G01N 33/5091; G01N 2800/52; G01N 21/77; G01N 2800/7009; G01N 2800/7028; G01N 21/76

(57) ABSTRACT

This document provides methods and materials for assessing hydrogen peroxide accumulation within cells (e.g., cancer cells) exposed to one or more test agents. For example, methods and materials for determining whether or not cancer cells (e.g., MM cells) from a mammal (e.g., a human) accumulate hydrogen peroxide following contact with a test agent (e.g., an IMID) and exogenous H₂O₂ are provided.

### CROSS-REFERENCE TO RELATED APPLICATIONS

This application claims priority to U.S. Application Ser. No. 62/180,327, filed on Jun. 16, 2015. The disclosure of the prior application is considered part of the disclosure of this application, and is incorporated in its entirety into this application.

### BACKGROUND

### 1. Technical Field

This document relates to methods and materials for assessing hydrogen peroxide accumulation within cells exposed to one or more test agents. For example, this document provides methods and materials for determining whether or not cancer cells from a mammal accumulate hydrogen peroxide following contact with a test agent.

### 2. Background Information

Multiple myeloma (MM) is a cancer that develops from the malignant proliferation of plasma cells in the bone marrow. MM can be treated with an immunomodulatory drug (IMID), which may be administered alone or in combination with other agents such as proteasome inhibitors.

### SUMMARY

This document provides methods and materials for assessing hydrogen peroxide accumulation within cells (e.g., cancer cells) exposed to one or more test agents. For example, this document provides methods and materials for determining whether or not cancer cells (e.g., MM cells) from a mammal (e.g., a human) accumulate hydrogen peroxide following contact with a test agent (e.g., an IMID) and exogenous H₂O₂. As described herein, cancer cells in solution can be exposed to a test agent and exogenous H₂O₂. Following this exposure, the solution containing the cells can be examined (e.g., visually examined) for the presence, absence, or level of O₂ or bubble formation. Those test agents that result in the formation of detectable (e.g., visually detectable) O₂ or bubbles can be identified as being a test agent with little or no treatment potential for the mammal from which the cells were obtained. Those test agents that result in the absence of or minimal formation of detectable (e.g., visually detectable) O₂ or bubbles can be identified as being a test agent with treatment potential for the mammal from which the cells were obtained. In some cases, a particular IMID such as lenalidomide can be identified as having little or no treatment potential in one human based on the formation of detectable (e.g., visually detectable) O₂ or bubbles using that human's cancer cells, while that same IMID (e.g., lenalidomide) can be identified as having treatment potential in a different human based on the lack of formation of detectable (e.g., visually detectable) O₂ or bubbles using that different human's cancer cells. Once identified as having treatment potential for a particular mammal (e.g., a particular human), then that identified agent can be administered to that particular mammal to treat cancer within that mammal.

Having the ability to contact cells (e.g., cancer cells) with a test agent and exogenous H₂O₂ and then to inspect (e.g., inspect visually) the solution containing those cells for evidence of O₂ or bubble formation to determine whether or not the test agent results in an accumulation of hydrogen peroxide within the cells can allow clinicians and patients to identify appropriate treatment options (e.g., an effective IMID for that particular patient) in a quick and accurate manner. In some cases, quantification of H₂O₂ mediated oxidized FAD and NAD(P) can be used in a manner that is more quantitative and accurate to determine cellular anti-oxidative capacity than O₂ bubbles.

In general, one aspect of this document features a method for measuring H₂O₂ accumulation in a cell following exposure to an agent. The method comprises, or consists essentially of, (a) contacting cells in a solution with the agent and exogenous H₂O₂; and (b) determining whether or not O₂ forms in the solution, wherein formation of the O₂ in the solution indicates that the agent does not cause accumulation of H₂O₂ in the cells, and wherein a lack of formation of the O₂ in the solution indicates that the agent causes accumulation of H₂O₂ in the cells. The agent can be an IMID. The IMID can be thalidomide, lenalidomide, pomalidomide, or apremilast. The exogenous H₂O₂ can be provided in an amount from about 20 μM to about 150 μM. The exogenous H₂O₂ can be provided in an amount of about 100 μM. The solution can be phosphate buffered saline (PBS). The cells can be cancer cells. The cancer cells can be myelodysplastic syndrome cells, erythema nodosum leprosum cells, multiple myeloma cells, Hodgkin's lymphoma cells, light chain-associated amyloidosis cells, primary myelofibrosis cells, acute myeloid leukaemia cells, prostate cancer cells, or metastatic renal cell carcinoma cells. The determining whether or not O₂ forms in the solution can comprise (i) determining whether or not bubbles form in the solution, (ii) determining whether or not autofluorescence of FAD increases, or (iii) determining whether or not autofluorescence of NAD(P)H decreases. The determining step can comprise visually detecting formation of the bubbles.

In another aspect, this document features a method for identifying a cancer treatment agent for a mammal having cancer. The method comprises, or consists essentially of, (a) obtaining cancer cells from the mammal; (b) contacting the cells in a solution with a test agent and exogenous H₂O₂; and (c) detecting the absence of O₂ formation in the solution; wherein the absence of the O₂ formation in the solution indicates that the test agent is a potential cancer treatment agent for the mammal. The mammal can be a human. The agent can be an IMID. The IMID can be thalidomide, lenalidomide, pomalidomide, or apremilast. The exogenous H₂O₂ can be provided in an amount from about 20 μM to about 150 μM. The exogenous H₂O₂ can be provided in an amount of about 100 μM. The solution can be PBS. The cancer cells can be myelodysplastic syndrome cells, erythema nodosum leprosum cells, multiple myeloma cells, Hodgkin's lymphoma cells, light chain-associated amyloidosis cells, primary myelofibrosis cells, acute myeloid leukaemia cells, prostate cancer cells, metastatic renal cell carcinoma cells, or metastatic renal cell carcinoma cells. The detecting the absence of O₂ formation in the solution can comprise (i) detecting the absence of bubble formation in the solution, (ii) detecting the absence of an increase in autofluorescence of FAD, or (iii) detecting the absence of a decrease in autofluorescence of NAD(P)H. The detecting step can comprise visually detecting the absence of formation of the bubbles.

In another aspect, this document features a method for treating cancer, wherein the method comprises, or consists essentially of, (a) obtaining cancer cells from a mammal having cancer; (b) contacting the cancer cells in a solution with an agent and exogenous H₂O₂; (c) detecting an absence of O₂ formation in the solution; and (d) administering the agent to the mammal under conditions wherein the number of cancer cells within the mammal is reduced. The mammal can be a human. The agent can be an IMID. The IMID can be thalidomide, lenalidomide, pomalidomide, or apremilast. The exogenous H₂O₂ can be provided in an amount from about 20 μM to about 150 μM. The exogenous H₂O₂ can be provided in an amount of about 100 μM. The solution can be PBS. The cancer can be myelodysplastic syndrome, erythema nodosum leprosum, multiple myeloma, Hodgkin's lymphoma, light chain-associated amyloidosis, primary myelofibrosis, acute myeloid leukaemia, prostate cancer, or metastatic renal cell carcinoma cells. The detecting the absence of O₂ formation in the solution can comprise (i) detecting the absence of bubble formation in the solution, (ii) detecting the absence of an increase in autofluorescence of FAD, or (iii) detecting the absence of a decrease in autofluorescence of NAD(P)H. The detecting step can comprise visually detecting the absence of formation of the bubbles.

In another aspect, this document features a method for treating cancer, wherein the method comprises, or consists essentially of, (a) obtaining cancer cells from a mammal having cancer; (b) contacting at least a portion of the cancer cells in a first solution with a first agent and exogenous H₂O₂; (c) detecting the presence of O₂ formation in the first solution; (d) contacting at least a portion of the cancer cells in a second solution with a second agent and exogenous H₂O₂; (e) detecting the absence of O₂ formation in the second solution; and (0 administering the second agent to the mammal under conditions wherein the number of cancer cells within the mammal is reduced. The mammal can be a human. The first agent can be an IMID. The second agent can be an IMID. The IMID can be thalidomide, lenalidomide, pomalidomide, or apremilast. The exogenous H₂O₂ can be provided in an amount from about 20 μM to about 150 μM. The exogenous H₂O₂ can be provided in an amount of about 100 μM. The method of claim 31, wherein the first solution can be phosphate buffered saline (PBS), and wherein the second solution is PBS. The cancer can be myelodysplastic syndrome, erythema nodosum leprosum, multiple myeloma, Hodgkin's lymphoma, light chain-associated amyloidosis, primary myelofibrosis, acute myeloid leukaemia, prostate cancer, or metastatic renal cell carcinoma. The detecting the presence of O₂ formation in the first solution can comprise (i) detecting the presence of bubble formation in the first solution, (ii) detecting an increase in autofluorescence of FAD in the first solution, or (iii) detecting a decrease in autofluorescence of NAD(P)H in the first solution. The detecting the absence of O₂ formation in the second solution can comprise (i) detecting the absence of bubble formation in the second solution, (ii) detecting the absence of an increase in autofluorescence of FAD in the second solution, or (iii) detecting the absence of a decrease in autofluorescence of NAD(P)H in the second solution. The detecting steps can comprise visually detecting the presence or absence of formation of the bubbles.

In another aspect, this document features a method for treating cancer, wherein the method comprises, or consists essentially of, (a) obtaining cancer cells from a mammal having cancer; (b) placing a portion of the cancers into a plurality of different containers in solution; (c) adding a different test agent to each of the plurality of different containers; (d) adding exogenous H₂O₂ to each of the plurality of different containers; (e) detecting the level of O₂ formation in the solution of each of the plurality of different containers; (f) selecting the test agent present in one of the plurality of different containers that resulted in minimal O₂ formation as compared to the level observed in at least one other of the plurality of different containers, thereby identifying the selected test agent as a treatment agent for the mammal; and (g) administering the treatment agent to the mammal under conditions wherein the number of cancer cells within the mammal is reduced. The mammal can be a human. At least one of the test agents can be an IMID. The IMID can be thalidomide, lenalidomide, pomalidomide, or apremilast. The exogenous H₂O₂ can be provided in an amount from about 20 μM to about 150 μM. The exogenous H₂O₂ can be provided in an amount of about 100 μM. The solution can be PBS. The detecting the level of O₂ formation in the solution of each of the plurality of different containers can comprise (i) detecting the level of bubble formation in the solution of each of the plurality of different containers, (ii) detecting the level of autofluorescence of FAD in the solution of each of the plurality of different containers, or (iii) detecting the level of autofluorescence of NAD(P)H in the solution of each of the plurality of different containers. The detecting step can comprise visually detecting the level of formation of the bubbles.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 shows lenalidomide generated H₂O₂-mediated intracellular oxidative stress in MM cells. A, FACS analysis of MM.1S cells with or without 30-minute pretreatment with DCDFDA, 1 PBS wash, and treatment with lenalidomide or control DMSO (10,000 cells gates). No difference was observed with DCDFDA-untreated control cells. DCDFDA-pretreated cells exposed to lenalidomide had increased DCF fluorescence. B, Mean fluorescence intensity shown from 3 independent experiments. Error bars represent standard deviation. C, IMIDs inhibit Horseradish peroxidase mediated decomposition of H₂O₂ in-vitro. Thalidomide, Lenalidomide and Pomalidomide 10 μM concentration or control DMSO incubated with HRP Amplex Red and H₂O₂ (5 μM) for 30 minutes and fluorescence intensity was measured. D, IMIDs inhibit intracellular H₂O₂ decomposition in HMCLs. Different HMCLs were pre-treated with Amplex Red (50 μM) and then treated with Thalidomide (20 μM), Lenalidomide (20 μM) and Pomalidomide (10 μM) or control DMSO for 40 to 60 minutes and found that all IMIDs inhibited intracellular H₂O₂ decomposition by peroxidases. E, IMIDs inhibit extracellular H₂O₂ decomposition in HMCLs. Different HMCLs were pre-treated with Amplex Red (50 μM) and then treated with H₂O₂ (100 μM) alone or in combination with different IMIDs Thalidomide (20 μM), Lenalidomide (20 μM) and Pomalidomide (10 μM) for 30 minutes and analyzed for resorufin fluorescence. F, MM.1S cells were treated with DMSO (control) and lenalidomide (10 μM) for different intervals. Cell lysates were prepared, separated by electrophoresis under nonreducing conditions (without DTT), and immunoblotted as indicated. DCDFDA indicates 2′,7′-dichlorodihydrofluorescein diacetate; DCF, 2′,7′-dichlorofluorescein; DMSO, dimethyl sulfoxide; DTT, dithiothreitol; FACS, fluorescence-activated cell sorting; GAPDH, glyceraldehyde-3-phosphate dehydrogenase; GSH, reduced glutathione; Len, lenalidomide; PBS, phosphate-buffered saline.

FIG. 2 shows IMIDs inhibit H₂O₂ decomposition in HMCLs and induce IKZF1 protein dimerization. A, HMCLs (KMS11 and JJN3) were pre-treated with Amplex Red (50 μM) and then treated with thalidomide (20 μM), lenalidomide (20 μM) and pomalidomide (10 μM), as control DMSO for 40 minutes or in addition with 100 μM H₂O₂ for 30 minutes and analyzed for resorufin fluorescence. B, MM.1S cells were treated with lenalidomide (10 μM) and H₂O₂ (25 or 50 μM) for 60 minutes. Protein lysates were prepared and separated by electrophoresis under reducing or nonreducing conditions (with or without DTT), and immunoblotted as indicated to evaluate IKZF1 dimerization. DMSO indicates dimethyl sulfoxide; DTT, dithiothreitol; FACS, fluorescence-activated cell sorting; GAPDH, glyceraldehyde-3-phosphate dehydrogenase; Len, lenalidomide; PBS, phosphate-buffered saline.

FIG. 3 shows H₂O₂ leads to preferential degradation of IKZF1 and IKZF3 in CRBN-positive cells. A, MM.1S cell line was treated with lenalidomide (10 μM), etoposide (1 μM), doxorubicin (0.5 μM), H₂O₂ (100 μM), and dexamethasone (20 μM) for 3 hours. Cell lysates were prepared, separated by electrophoresis, and immunoblotted with indicated antibodies. B, OPM2-nontarget (control) and OPM2-shCRBN (silencing) were treated with lenalidomide (10 μM) or increasing concentrations of H₂O₂ (25 or 50 μM) for 3 hours. Protein lysates were prepared, separated by electrophoresis, and immunoblotted as indicated. C, OPM2-NT and OPM2-shCRBN were treated with increasing concentrations of H₂O₂ for 3 days. MTT assays were performed and cell survival was plotted. D, OPM2-NT and OPM2-shCRBN were treated with increasing concentrations of lenalidomide for 3 days. MTT assays were performed and cell survival was plotted. E, OCIMY-5 cells overexpressing CRBN and control vector were treated with lenalidomide (10 μM) and increasing concentrations of H₂O₂ (25 or 50 μM) for 3 hours. Protein lysates were prepared, separated by electrophoresis, and immunoblotted as indicated. F, OCIMY-5 cells overexpressing CRBN and control vector were treated with increasing concentrations of H₂O₂ for 3 days. MTT assays were performed and cell survival was plotted. G, OCIMY-5 cells overexpressing CRBN and control vector were treated with increasing concentrations of lenalidomide for 3 days. MTT assays were performed and cell survival was plotted. Cont indicates control; CRBN, cereblon; Dexa, dexamethasone; Doxo, doxorubicin; Eto, etoposide; Len, lenalidomide; MTT, 3-(4,5-dimethylthiazol-2-yl)-2,5-diphenyltetrazolium bromide; NT, nontarget; sh, short hairpin.

FIG. 4 shows H₂O₂ induced degradation of IKZF1 in CRBN-overexpressing OCIMY-5 Cells. Cells overexpressing CRBN or the control vector were treated with lenalidomide (10 μM) or H₂O₂ (50 μM) for 3 hours. Protein lysates were prepared, separated by electrophoresis, and immunoblotted as indicated.

FIG. 5 shows myeloma cells with less antioxidative capacity are highly vulnerable to lenalidomide-mediated cytotoxicity. A, MTT assay of MM cell lines showed that MM.1S was lenalidomide sensitive and RPMI-8226 was lenalidomide resistant. B, Rate of H₂O₂ decomposition by HMCLs corresponded with lenalidomide sensitivity. More oxygen bubbles after H₂O₂ treatment was associated with high antioxidative capacity and resistance to lenalidomide. See also FIG. 6. C, MM.1S and RPMI-8226 were seeded and incubated with H₂O₂ at the indicated concentrations for 3 days and MTT assays were performed. Each experimental condition was assayed in triplicate and repeated at least once. D, MM.1S and RPMI-8226 cell lines were treated with the indicated concentration of lenalidomide and H₂O₂ for 3 hours. Cell lysates were prepared, separated by electrophoresis, and immunoblotted with indicated antibodies. HMCL indicates human multiple myeloma cell line; Len, lenalidomide; MTT, 3-(4,5-dimethylthiazol-2-yl)-2,5-diphenyltetrazolium bromide. E-G, 100 μM H₂O₂ treatment increased oxidized FAD and NAD(P), which were quantitatively determined by FACS to correlate with anti-oxidative capacity and were used to determine anti-oxidative capacity.

FIG. 6 shows an association between antioxidative capacity and lenalidomide resistance. A, Different multiple myeloma cell lines with varying sensitivity to lenalidomide were examined. An equal number of cells in equal volume were treated with equal amounts of H₂O₂; the amount of oxygen bubbles formed were qualitatively examined and it's correlated with lenalidomide sensitivity. Greater lenalidomide resistance corresponded to greater oxygen bubble formation. B, RPMI-8226, JJN3, MM.1S, and KMS11 cells were seeded and incubated with lenalidomide at the indicated concentration for 3 days. MTT assays were performed and cell survival was plotted. C, RPMI-8226, JJN3, MM.1S, and KMS11 cells lysates were prepared, separated by electrophoresis, and immunoblotted with the indicted antibodies for showing basal protein levels of CRBN, IKZF1 and IKZF3 are not correlating with lenalidomide sensitivity.

FIG. 7 shows lenalidomide-induced oxidative stress causes IgL dimerization. A, MM.1S cell line was treated with lenalidomide (10 μM) or H₂O₂ (25 μM) for 3 hours. Cell lysates were prepared, separated by electrophoresis, and immunoblotted as indicated. For analyzing IgL homodimerization, proteins were separated under nonreducing conditions (without DTT). B, Lenalidomide-sensitive (MM.1S) and lenalidomide-resistant (MM.1S.res) cell lines were treated with lenalidomide for 3 days. Cell lysates were prepared under nonreducing conditions; protein lysates were separated by electrophoresis under nonreducing conditions (without DTT), and immunoblotted as indicated. C, MM.1S and MM.1S.res cells were seeded and incubated with lenalidomide at the indicated concentration for 3 days and MTT assays were performed. Each experimental condition was performed in triplicate and repeated at least once. D-F, CRBN-knockdown OPM2 and KMS18 cell lines, CRBN-overexpressing OCIMY5, and vector control cells were treated with lenalidomide for 3 days. Cell lysates were both prepared and separated by electrophoresis under nonreducing conditions (without DTT) to detect IgL dimerization. Immunoblots were performed as indicated. See also FIG. 8A. G-I, CRBN-positive cells were more sensitive to lenalidomide than CRBN-negative cells, as measured by MTT assay. CRBN indicates cereblon; DMSO, dimethyl sulfoxide; DTT, dithiothreitol; IgL, immunoglobulin light chain; Len, lenalidomide; MTT, 3-(4,5-dimethylthiazol-2-yl)-2,5-diphenyltetrazolium bromide.

FIG. 8 shows lenalidomide induced IgL-dimer accumulation induce ER-stress in MM. A, OPM2-NT and OPM2-shCRBN were treated with lenalidomide (10 μM) for 72 hours. Protein lysates were separated by electrophoresis under reducing or non-reducing conditions (with or without DTT) and immunoblotted as indicated. B, OPM2 NT and OPM2 shCRBN cells were treated with increasing concentrations of lenalidomide (10, 30, or 50 μM) for 72 hours. RT-PCR for IgL-λ was performed on prepared cDNA. Although lenalidomide treatment was associated with accumulated IgL-λ protein in CRBN-positive cells, it was not due to mRNA overexpression. C, OPM2 NT and shCRBN cells were treated for 72 hours with increasing concentrations of lenalidomide (10, 30, or 50 μM) or DMSO (control). Cell lysates were prepared, separated by electrophoresis, and immunoblotted with the indicated antibodies. β-actin was used as loading control. D, OPM2 NT and shCRBN cells were treated with increasing concentrations of lenalidomide for 2 days. Live cells were counted, and 1 million cells were plated in 4 mL of optimum medium with different concentrations of lenalidomide or DMSO (control). After 5 hours, samples were harvested by centrifugation. Supernatant (50 μL) proteins were separated by electrophoresis, and immunoblotted with anti-IgL-λ antibodies. Medium contain bovine serum albumin was used as a loading control. E, OPM2-NT and OPM2-shCRBN knockdown cells were incubated with lenalidomide (10 μM) for 48 hours. Secreted and intracellular IgL-λ levels were determined via enzyme-linked immunosorbent assay. Data are expressed as a percentage of control (mean±SD, n=3). CRBN indicates cereblon; DTT, dithiothreitol; IgL, immunoglobulin light chain; NT, nontarget; sh, short hairpin.

FIG. 9 shows lenalidomide-generated H₂O₂ induced IgL dimerization and mediated ER stress. A, OPM2-NT and CRBN knockdown (shCRBN) cells were treated for 3 days with increasing concentration of lenalidomide (10, 30, or 50 μM). Cell lysates were prepared, separated by electrophoresis, and immunoblotted as indicated. Lenalidomide induced intracellular IgL accumulation and ER stress in CRBN-positive cells but not CRBN-negative cells. B, Lenalidomide treatment induced XBP-1 mRNA splicing in CRBN cells. OPM2-NT and shCRBN cells were treated with lenalidomide (10 μM) for 3 days. RT-PCR was performed to evaluate XBP-1 mRNA splicing, a marker of ER stress. Lenalidomide induced XBP-1 mRNA splicing in CRBN-positive cells but not shCRBN cells. C, OPM2-NT and shCRBN cells were treated for 6 days with increasing concentration of lenalidomide (10, 30, and 50 μM). Cell lysates were prepared, separated by electrophoresis, and immunoblotted as indicated. Lenalidomide-induced, ER stress-mediated p53 degradation and PARP were more prominent at day 6 in OPM2 NT cells compared with shCRBN cells. D, OCIMY5 cells overexpressing wild type CRBN progressively accumulated IgL dimers after lenalidomide treatment and induced ER stress. OCIMY5-vector and CRBN cells were treated with lenalidomide (20 μM) for different periods (24-96 hours). Cell lysates were prepared, separated by electrophoresis, and immunoblotted as indicated. E, OPM2 cells stably expressing either piLenti-siRNA-GFP (scramble) or piLenti-si-IgL-λ-GFP (si-λ) were seeded and incubated with lenalidomide at the indicated concentration for 3 days and MTT assays were performed. Each experimental condition was performed in triplicate and repeated at least twice. See also FIG. 10. F, IgL-λ knockdown and control cells were treated with lenalidomide (10 μM) for 3 days. Cell lysates were prepared, separated by electrophoresis under nonreducing conditions, and immunoblotted as indicated. β-actin was used as the loading control. H929, IgL-κ knockdown by siRNA induced resistance to lenalidomide. G, Both IgL-κ knockdown and scramble siRNA control were treated with increasing concentrations of lenalidomide for 3 days and MTT assays were performed. H, IgL-κ knockdown and controls were treated with lenalidomide (10 μM) for 3 days. Protein lysates were prepared, separated by electrophoresis under nonreducing conditions, and immunoblotted as indicated. CRBN indicates cereblon; ER, endoplasmic reticulum; his, polyhistidine tag; IgL, immunoglobulin light chain; MTT, 3-(4,5-dimethylthiazol-2-yl)-2,5-diphenyltetrazolium bromide; NT, nontarget; PARP, poly (ADP-ribose) polymerase; RT-PCR, real-time polymerase chain reaction; sh, short hairpin; si, small interfering; wt, wild type; XBP-1s/u, XPB-1 spliced/unspliced.

FIG. 10 shows IgL knock-down mediate lenalidomide resistance in MM cells. A, OPM2 cells stably expressing NT-shRNA (OPM2-Vectotr) or OPM2-sh-λ were seeded and incubated with lenalidomide at the indicated concentrations for 3 days and MTT assays were performed. Each experimental condition was performed in triplicate and repeated at least twice. B, OPM2 IgL-λ knockdown (sh-λ) and control (GPZ) cells were treated with lenalidomide (10 μM) for 3 days. Cell lysates were prepared, separated by electrophoresis under nonreducing conditions, and immunoblotted using the indicated antibodies. IgL, immunoglobulin light chain; MTT, 3-(4,5-dimethylthiazol-2-yl)-2,5-diphenyltetrazolium bromide; NT, nontarget; sh, short hairpin.

FIG. 11 shows lenalidomide-induced ER stress triggers cytotoxicity by activating BH3 protein Bim in multiple myeloma. A, OPM2-NT and OPM2-shCRBN cells were treated with increasing concentrations of lenalidomide (10, 30, and 50 μM) for 72 hours. Cell lysates were prepared, separated by electrophoresis, and immunoblotted as indicated. The proapoptotic BH3-only protein Bim isoforms predominantly accumulated after lenalidomide treatment in CRBN-positive cells. B, Additional CRBN-positive and CRBN-negative isogenic cell lines (OCIMY5 CRBN overexpressing, KMS18 CRBN knockdown, and MM.1S sensitive and resistant to lenalidomide were treated with lenalidomide for 3 days. Cell lysates were prepared and immunoblotted with indicated antibodies. Bim activation was predominantly mediated by lenalidomide in CRBN-positive cells but not in CRBN-negative or CRBN knockdown cells. C, Knockdown of Bim-mediated resistance to lenalidomide. Bim shRNA-expressing stable OPM2 clones #73, #75, and NT control cells were treated with different concentrations of lenalidomide for 4 days and cell viability was determined with MTT assay with respect to DMSO-treated control. D, OPM2-NT and OPM2-shBim clones #73 and #75 were treated with or without lenalidomide (10 μM) for 72 hours. Cell lysates were prepared, separated by electrophoresis, and immunoblotted as indicated. Bim was down-regulated in the clones with respect to the NT control, and this pattern was maintained even after lenalidomide treatment increased Bim protein levels. Total PARP protein level was lower in NT cells compared with the Bim knockdown clones. β-Actin was used as the loading control. CRBN indicates cereblon; ER, endoplasmic reticulum; NT, nontarget; PARP, poly (ADP-ribose) polymerase; sh, short hairpin.

FIG. 12 shows pretreatment with lenalidomide enhanced bortezomib sensitivity in multiple myeloma. A, OPM2 cells, with or without 2 days of pretreatment with lenalidomide (10 μM), were treated with bortezomib (2.5 nM), lenalidomide (10 μM), and bortezomib+lenalidomide for an additional 2 days. Samples were collected and stained with annexin V-FITC and propidium iodide. FACS analysis for apoptosis showed the lowest viability with lenalidomide pretreatment followed by bortezomib+lenalidomide. B, Pretreatment with lenalidomide enhances bortezomib-mediated apoptosis in CRBN-positive myeloma cells. OPM2-NT and OPM2-shCRBN cells, with or without pretreatment with lenalidomide for 2 days, were treated with bortezomib (2.5 nM) for an additional 2 days. Cell lysates were prepared, separated by electrophoresis, and immunoblotted as indicated. Lenalidomide pretreatment enhanced bortezomib-induced apoptosis of CRBN-positive cells but had a less pronounced effect in CRBN-knockdown cells. CRBN indicates cereblon; FACS, fluorescence-activated cell sorting; FITC, fluorescein isothiocyanate; Len, lenalidomide; PARP, poly (ADP-ribose) polymerase; Pre, pretreatment.

FIG. 13 shows schematic representation of lenalidomide activity in MM. MM cells overproduce immunoglobulins, which generate high quantities of H₂O₂ through intramolecular and intermolecular disulfide bond formation. Additionally, lenalidomide-bound CRBN also causes intracellular generation of H₂O₂. Cells with high antioxidative capacity are resistant to apoptosis from H₂O₂-mediated oxidative stress. For cells with lower antioxidative capacity, this stress leads to the degradation of IKZF1, IKZF3, and p53, immunoglobulin dimerization, and subsequent ER stress-mediated, Bim-dependent apoptosis. CRBN indicates cereblon; ER, endoplasmic reticulum; Ig, immunoglobulin; IgL, immunoglobulin light chain; MM, multiple myeloma.

FIG. 14 contains graphs showing the use of resorufin fluorescence following exposure of cells to amplex red and exogenous H₂O₂ to identify cells that are sensitive or resistant to IMIDs (e.g., lenalidomide).

FIG. 15 contains graphs showing the use of NAD(P)H and FADH₂ autofluorescence to assess the anti-oxidative capacity of cells following exogenous H₂O₂ exposure.

FIGS. 16A-D are graphs showing anti-oxidative capacity. External H₂O₂ exposure induced high oxidation of FADH₂ (FIG. 16A) and NAD(P)H (FIG. 16B). Pre-treatment with lipoic acid or lopoamide decreased myeloma cells anti-oxidative capacity of FADH₂ (FIG. 16C) and NAD(P)H (FIG. 16D).

FIG. 17 contains graphs plotting cell viability results using an MTT assay for OCIMY5-Vector and OCIMY-CRBN cells treated with indicated concentration of the indicated drugs for 72 hours.

FIG. 18 contains graphs plotting cell viability results using an MTT assay for lenalidomide sensitive and lenalidomide resistant cells treated with indicated concentration of the indicated drugs for 72 hours.

FIG. 19 contains graphs plotting cell viability results using an MTT assay for the indicated cells treated with the indicated concentrations of either auranofin or lenalidomide.

FIG. 20 contains graphs plotting cell viability results using an MTT assay for the indicated cells treated with the indicated concentrations of auranofin.

FIG. 21 contains a schematic and a graph plotting cell viability results using an MTT assay for the indicated cells treated with the indicated concentrations of dehydroascorbic acid.

FIG. 22 contains graphs plotting cell viability results using an MTT assay for myeloma cell line harboring CRBN and myeloma cell line without CRBN treated with the indicated compound or combination of compounds.

FIG. 23 contains graphs plotting percent viability for the indicated myeloma cell line harboring CRBN and myeloma cell line without CRBN treated with auranofin alone or auranofin in combination with the indicated concentration of bortezomib.

FIG. 24 is a photograph of a western blot using a MM.1S cell line treated with lenalidomide (10 μM), aurothiomalate (10 μM), or DMSO (control) for 3 hours. Protein lysates were prepared and blotted with the indicated antibodies.

FIG. 25 contains photographs of western blots using MM.1S and OPM2 cells treated with lenalidomide (10 μM), PX12 (20 μM), or DMSO (control) for 3 hours. Protein lysates were prepared, separated by electrophoresis, and immunoblotted with the indicated antibodies. Blots are representative of three independent experiments.

### DETAILED DESCRIPTION

This document provides methods and materials for assessing hydrogen peroxide accumulation within cells (e.g., cancer cells) exposed to one or more test agents. For example, this document provides methods and materials for determining whether or not cancer cells (e.g., MM cells) from a mammal (e.g., a human) accumulate hydrogen peroxide following contact with a test agent (e.g., an IMID) and exogenous H₂O₂.

In some cases, cells (e.g., cancer cells) in solution can be exposed to a test agent and exogenous H₂O₂. If the cells (e.g., cancer cells) possess the ability to degrade H₂O₂ even in the presence of the test agent, then water and O₂ will be formed. This O₂ can be detected, thereby providing an indication that little to no H₂O₂ is accumulating within those particular cells (e.g., cancer cells) when contacted with the test agent. If the cells (e.g., cancer cells) lack the ability to degrade H₂O₂ in the presence of the test agent, then water and O₂ will not be formed from the added H₂O₂. This lack of O₂ can be detected, thereby providing an indication that H₂O₂ is accumulating within those particular cells (e.g., cancer cells) when contacted with the test agent.

Those test agents having the ability to prevent or reduce O₂ formation from added H₂O₂ for particular cancer cells can be used as a cancer treatment agents. For example, a test agent found to have the ability to prevent the formation of visible bubbles from added H₂O₂ by cancer cells obtained from a particular human cancer patient can be administered to that particular patient to treat cancer.

Any appropriate method can be used to detect the formation of O₂ from added H₂O₂. For example, measuring increases in autofluorescence of FAD (FITC fluorescence spectrum) and/or measuring decreases in autofluorescence of NAD(P)H (UV-blue fluorescence spectrum), which can correlate with O₂ bubble formation, can be used to measure O₂ formation within a solution. In some cases, the solution containing the cells can be examined (e.g., visually examined) for the presence, absence, or level of bubble formation (e.g., formation of O₂-containing bubbles).

Any appropriate compound can be used as a test agent. For example, IMIDs can be used as a test agent. Examples of IMIDs include, without limitation, thalidomide, lenalidomide, pomalidomide, and apremilast.

The methods and materials provided herein can be used with any appropriate cell. For example, exogenous H₂O₂ and a test agent can be added to cancer cells in solution to determine if those cancer cells have the ability degrade or accumulate H₂O₂. Examples of cancer cells that can be used include, without limitation, myelodysplastic syndrome cells, erythema nodosum leprosum cells, multiple myeloma cells, Hodgkin's lymphoma cells, light chain-associated amyloidosis cells, primary myelofibrosis cells, acute myeloid leukaemia cells, prostate cancer cells, and metastatic renal cell carcinoma cells. Any appropriate number of cells can be used. For example, from about 1×10⁴ to about 1×10⁹ cells (e.g., from about 1×10⁵ to about 1×10⁹ cells, from about 1×10⁶ to about 1×10⁹ cells, from about 1×10⁷ to about 1×10⁹ cells, from about 1×10⁸ to about 1×10⁹ cells, from about 1×10⁵ to about 1×10⁸ cells, or from about 1×10⁶ to about 1×10⁸ cells) per mL of solution can be contacted with exogenous H₂O₂ and a test agent. Any appropriate amount of exogenous H₂O₂ and test agent can be used. For example, from about 0.001 mM to about 10 M (e.g., from about 0.001 mM to about 5 M, from about 0.001 mM to about 1 M, from about 0.001 mM to about 750 mM, from about 0.001 mM to about 500 mM, from about 0.001 mM to about 250 mM, from about 0.001 mM to about 100 mM, from about 0.001 mM to about 50 mM, from about 0.001 mM to about 25 mM, from about 0.01 mM to about 750 mM, from about 0.05 mM to about 750 mM, from about 0.1 mM to about 750 mM, from about 1 mM to about 750 mM, from about 1 mM to about 50 mM, or from about 1 mM to about 10 mM) of H₂O₂ can be used. In some cases, from about 0.001 mM to about 10 mM of test agent can be used.

The cells can be contacted with exogenous H₂O₂ and a test agent in any appropriate solution. For example, cells within PBS can be contacted with exogenous H₂O₂ and a test agent, and the solution assessed for O₂ production and/or bubble formation.

Once the cells are contacted with exogenous H₂O₂ and a test agent, the solution can be assessed after about 1 minute to about 180 minutes (e.g., after about 5 minute to about 180 minutes, after about 10 minute to about 180 minutes, after about 15 minute to about 180 minutes, after about 25 minute to about 180 minutes, after about 60 minute to about 180 minutes, after about 5 minute to about 120 minutes, after about 5 minute to about 75 minutes, after about 5 minute to about 60 minutes, after about 10 minute to about 75 minutes, after about 10 minute to about 60 minutes, or after about 25 minute to about 60 minutes) for the presence, absence, or level of O₂ formation and/or bubble formation (e.g., formation of O₂-containing bubbles).

In some cases, the methods and materials provided herein can be used to assess IMID responsiveness in a mammal to be treated for cancer. Accumulation of intracellular H₂O₂ produced by an IMID can lead to apoptosis. Cells, however, may have an antioxidant defense system to decompose H₂O₂, thereby combating excessive production of H₂O₂. A cell's ability to decompose H₂O₂ is referred to as its anti-oxidative capacity and is indicative of IMID responsiveness. A cell with higher anti-oxidative capacity (efficiently decomposes H₂O₂) is indicative of IMID resistance, and a cell with a lower anti-oxidative capacity (inefficiently decomposes H₂O₂) is indicative of IMID sensitivity.

In some cases, the methods and materials described herein can be used to grade cancers for low anti-oxidative capacity, medium anti-oxidative capacity, or high anti-oxidative capacity. This can allow clinicians to select different treatment strategies for particular patients. In some cases, determining the total anti-oxidative capacity of cancer cells during therapy can be used as a prognostic marker. For example, the methods and materials provided herein can be used to determine whether or not a mammal (e.g., a human) having cancer is responding to a particular IMID based at least in part on the anti-oxidative capacity of cancer cells obtained from the mammal at different treatment time points.

As described herein, exogenously added H₂O₂ can result in the production of water and oxygen when contacted with cells. The qualitative estimate of oxygen bubbles generated from H₂O₂ can indicate IMID responsiveness.

Any appropriate mammal can be assessed and/or treated as described herein. For example, humans, non-human primates, monkeys, horses, bovine species, porcine species, dogs, cats, mice, and rats having cancer can be assessed to determine whether or not the mammal is likely to respond to an IMID and/or likely to be treated for cancer with a particular agent. In some cases, a mammal having any appropriate type of cancer can be assessed and/or treated as described herein. For example, mammals with myelodysplastic syndrome, erythema nodosum leprosum, multiple myeloma, Hodgkin's lymphoma, light chain-associated amyloidosis, primary myelofibrosis, acute myeloid leukaemia, prostate cancer, and metastatic renal cell carcinoma can be assessed to determine whether or not the mammal is likely to respond to an IMID and/or is a candidate for a particular cancer treatment.

In some cases, the methods and materials provided herein can be used to identify agents that have the ability to increase intracellular H₂O₂ accumulation in cells (e.g., cancer cells). For example, a test agent can be incubated with cells in solution in the presence of exogenous H₂O₂, and the solution can be assessed as described herein for the presence of O₂ formation and/or bubble formation (e.g., increase oxidized FAD autofluorescence and/or decreased NAD(P)H autofluorescence). Those test agents that increase oxidized FAD autofluorescence and/or decreased NAD(P)H autofluorescence can be identified as being an agent that increases intracellular H₂O₂ accumulation.

In some cases, test agents can be assessed to determine if they inhibit intracellular H₂O₂ decomposition, thereby increasing intracellular H₂O₂. Test agents having this ability can be used for cancer therapy. Examples of agents having the ability to inhibit intracellular H₂O₂ decomposition include, without limitation, H₂O₂ analogues (D₂O₂ and HDO₂), glutathione peroxidase inhibitors including glutathione analogs, NADPH peroxidase inhibitors and NADPH analogues, catalase inhibitors, thioredoxin peroxidase inhibitors, haem peroxidase inhibitors, peroxidase substrates that inhibit H₂O₂ decomposition, homocysteine, cysteine analogs that can inhibit H₂O₂ decomposition, and hydrogen peroxide stabilizers including inorganic phosphate. In some cases, a test agent can be assessed for the ability to increase intracellular H₂O₂ production in cancer cells by altering cancer cell metabolism and/or promoting the production of intracellular H₂O₂. Examples of agents having the ability to increase intracellular production of H₂O₂ include, without limitation, mitochondrial respiration activators (e.g., lipoamide, (R)-(+)-α-lipoic acid, and (S)-(−)-α-lipoic acid), citrate, ATP, NADH, agents that induce fatty acid and lipid biosynthesis such as second-generation antipsychotics (SGA) (e.g., clozapine, olanzapine, and dihydrotestosterone), dexamethasone, 3-isobutyl-1-methylxanthine (ibmx), oxidized L-glutathione, malonyl-CoA, acetyl coenzyme A, coenzyme A, retinal (also called retinaldehyde), vitamin A aldehydes, and trans-retinals. Fatty-acid oxidation mediated generation of H₂O₂ can be achieved by supplementing with fatty acids that undergo rapid intracellular oxidation and generation of H₂O₂. In some cases, redox cycling compounds (e.g., pyrroloquinoline quinone (PQQ), anisaldehyde, and veratraldehyde) can be used to generate intracellular H₂O₂. In some cases, a combination of at least one agent having the ability to inhibit intracellular H₂O₂ decomposition and at least one agent having the ability to increase intracellular production of H₂O₂ can be administered to a mammal (e.g., a human) having cancer to reduce the number of cancer cells within the mammal.

As described herein, lenalidomide and other IMIDs can inhibit thioredoxin reductase and induce the accumulation of intracellular H₂O₂ in cancer cells (e.g., myeloma cells). In some cases, thioredoxin reductase inhibitors can be used to increase intracellular H₂O₂ in cancer cells (e.g., myeloma cells) and can be used alone or in combination with other anti-cancer drugs to treat cancer. Examples of thioredoxin reductase inhibitors that can be used to treat cancer as described herein include, without limitation, auranofin, aurothiomalate, alantolactone, phosphine gold(I), GoPI ({1-phenyl-2,5-di(2-pyridyl)phosphole}AuCl), gold(I)carbene complexes, gold(III)-dithiocarbamato complexes, AuBiPy, AuXil, AuPy, terpyridine-Pt(II), pyocyanin (5-methylphenazin-1(5H)-one), cisplatin (cis-diaminodichloroPt(II)), carboplatin, terpyridine-platinum(II), arsenic trioxide, methyl As(III), 2,4-Dihydroxybenzylamine, 13-cis retinoic acid, nitrosoureas, dinitrohalobenzenes, Ajoene ((E,Z)-4,5,9-trithiadodeca-1,6,11-triene 9-oxide), fluoro-analogue of a menadione derivative, bromo-isophosphoramide, peroxynitrite, dinitrosoglutathione, S-nitrosoglutathione, EGCG (epigallocatechin-3-O-gallate), n-butyl 2-imidazolyl disulfide, 1-methylpropyl 2-imidazolyl disulfide, n-decyl 2-imidazolyl disulfide, xanthene (6-hydroxy-3-oxo-3H-xanthene-9-propionic acid), and safranin (3,7-diamino-2,8-dimethyl-5-phenyl-phenazinium chloride).

In some cases, thioredoxin inhibitors can be used to increase intracellular H₂O₂ in cancer cells (e.g., myeloma cells) and can be used alone or in combination with other anti-cancer drugs to treat cancer. Examples of thioredoxin inhibitors that can be used to treat cancer as described herein include, without limitation, PX-12 (1-methylpropyl 2-imidazolyl disulfide), PMX464, DTNB (5,5′-dithiobis-(2-nitrobenzoic acid) and its analogs, and 4-hydroxy-2-nonenal.

The invention will be further described in the following examples, which do not limit the scope of the invention described in the claims.

### EXAMPLES

### Example 1—IMIDs Inhibit H₂O₂ Decomposition in Multiple Myeloma Cells and its Mediated Cytotoxicity is Determined by Cellular Antioxidative Capacity

### Cell Culture and MTT Assay

The human multiple myeloma cell line (HMCLs) OPM2, MM.1S, MM.1Sres, KMS18, JJN3, KMS11, and OCIMY5 were studied. All exhibited different degrees of sensitivity to lenalidomide. HMCLs were maintained in RPMI-1640, supplemented with 10% fetal bovine serum, 100 U/mL penicillin, 100 μg/mL streptomycin, and 2 mM glutamine. All HMCLs were grown at 37° C. in a 5% CO₂ incubator.

Cell viability was assessed by 3-(4,5-dimethylthiazol-2-yl)-2,5-diphenyltetrazolium bromide (MTT) colorimetric assay. Cells were seeded in 96-well plates in 100 μL complete medium at a density of 20,000 cell/well and incubated with serial doses of lenalidomide (Chem-Pacific), and bortezomib (millennium pharmaceuticals) for different periods. The data were normalized to the DMSO-treated group. Each experimental condition was performed in triplicate and repeated at least once. Thalidomide was obtained from Sigma, and pomalidomide was obtained from Selleckchem.

### Western Blot Analysis and Antibodies

Whole-cell lysates were prepared from cell pellets using cell lysis buffer (Cell Signaling Technology). Equal amounts of protein extracts were resolved by sodium dodecyl sulfate (SDS)-polyacrylamide gel electrophoresis. Proteins were transferred to polyvinylidene fluoride membranes (Bio-Rad). Most gels were run under reducing conditions (by adding dithiothreitol), but analysis of immunoglobulin light chain (IgL) dimers required non-reducing conditions (without dithiothreitol). After blocking with 5% milk for 1 hour at room temperature, membranes were washed and probed with primary antibodies overnight at 4° C. Blots were washed with 0.1% Tris-buffered saline and Tween 20 and incubated with appropriate horseradish peroxidase-labeled secondary antibodies. Blots were developed using a chemiluminescent detection system (ECL, PerkinElmer). β-actin or GADPH were used as loading control.

Antibodies included those against p53 (DO-1, Santa Cruz), XBP-1 (Santa Cruz), cereblon (CRBN) (Sigma), β-actin (Sigma), λ light chain, κ light chain (Abcam), Bip (Cell Signaling Technology), Bim (Cell Signaling Technology), and poly (ADP-ribose) polymerase (PARP) (Cell Signaling Technology). Other antibodies were obtained from Cell Signaling Technology. The secondary antibodies were horseradish peroxidase-conjugated goat anti-mouse and anti-rabbit immunoglobulin G (Cell Signaling Technology).

### Polymerase Chain Reaction

Total RNA from HMCLs treated with lenalidomide was isolated using the Qiagen RNeasy mini kit. RNA (1 μg) was used for cDNA synthesis using iScript Reverse Transcription Supermix (Bio-Rad). Real-time polymerase chain reaction (PCR) was performed using the SYBR green method. The following primers were used for amplification:

IGL-Lambda-F:
(SEQ ID NO: 1)
5′-GAGCCTDACGCCTGAG-3′
IGL-Lambda-R:
(SEQ ID NO: 2)
5′-ATTGAGGGTTTATTGAGTGCAG-3′
XBP-1-F:
(SEQ ID NO: 3)
5′-TTACGAGAGAAAACTCATGGC-3′
XBP-1-R:
(SEQ ID NO: 4)
5′-GGGTCCAAGTTGTCCAGAATG-3′
β-Actin-F:
(SEQ ID NO: 5)
5′-TAAAGACCTCTATGCCAACACAG-3′
β-Actin-R:
(SEQ ID NO: 6)
5′-CACGATGGAGGGGCCGGACTCATC-3′

The fold change of the mRNA expression was calculated from the difference between treated and untreated cells, after normalizing to an endogenous control (β-actin). Primers encompassing the spliced sequences in XBP1 mRNA were used for PCR amplification, and products were separated by electrophoresis through a 2.5% agarose gel and visualized by ethidium bromide staining. All reactions were conducted in triplicate.

### Apoptosis Assay

Treated cells were harvested and stained for flow cytometry with Annexin V-fluorescein isothiocyanate and propidium iodide (BD Pharmingen). Stained cells were analyzed with a BD LSRII flow cytometer, and the data were analyzed with BD fluorescence-activated cell sorting (FACs) DIVA software.

### CRBN, IgL-λ, and Bim Knockdown

CRBN knockdown cells were used as described elsewhere (Zhu et al., Blood, 118(18):4771-9 (2011)). For Bim knockdown, lentiviral constructs expressing non-targeting (shCtrl) and Bim short hairpin RNAs (shRNAs) (Sigma-Aldrich) were used. TRC-vectors were cotransfected into 293T cells using a calcium phosphate precipitation method with the psPAX2 packaging plasmid and pMD2.G, a plasmid encoding the lentivirus envelope. Supernatants containing pseudotyped lentivirus were collected at 48 and 72 hours and were used to infect HMCLs. Four lentiviruses targeting Bim were screened to identify shRNA that optimally suppressed Bim. Forty-eight hours after transfection, cells were selected with puromycin, and OPM2 lysates were immunoblotted to confirm down-regulation of Bim. Clones #73 and #75, which optimally suppressed Bim, were used for subsequent experiments. IgL-λ knockdown was performed by using piLenti-siRNA-GFP to target the constant region of IgL-λ. IgL-κ knockdown was performed by using lentiviral-mediated siRNA to target the constant region of IgL-κ.

### Augmented Ectopic Expression of CRBN in OCIMY5

Human CRBN cDNA was obtained from Thermo Scientific and subcloned into a lentiviral expression vector, pCDH-CMV-MCS-EF1-copGFP (System Bioscience). Lentivirus harboring control vector and CRBN cDNA constructs were prepared and used to infect the multiple myeloma (MM) cell line OCIMY5. Infection efficiency was measured by FACScan analysis of GFP expression 3 days after infection. The cells were sorted for GFP expression 14 days after infection. CRBN overexpression was confirmed by immunoblotting.

### Amplex Red Assay

HRP/Amplex Red in-vitro assay performed in 100 μL HBSS final reaction volume contain HRP (1 unit/mL) and Amplex Red (50 μM) and 10 μM concentration of drug (thalidomide, lenalidomide, and pomalidomide) or DMSO control with 5 μM concentration of H₂O₂ and kept for reaction at 37° C. for 30 minutes. After 30 minutes, plates were read for fluorescence at 530 nm excitation and 590 nm emission with plate reader (Biotek Cytation3). For determining intra-cellular peroxidase activity by Amplex Red, cells were washed and mixed with Amplex Red reagent (50 μM) in HBSS buffer and plated 100,000 cells per well into 96 well plate, four wells for each condition. After plating, the cells were immediately treated with H₂O₂ (100 μM) alone or together with thalidomide (20 μM), lenalidomide (20 μM), or pomalidomide (10 μM). The DMSO control included H₂O₂ (100 μM). The cells were incubated at 37° C. for 40 to 60 minutes for analyzing IMIDs ability to inhibit intracellular peroxidase activity and 30 minutes after H₂O₂ treatment for determining IMIDs ability to mediate inhibition of extracellular H₂O₂ decomposition. After specific time periods, the plates were read for fluorescence intensity using plate reader.

### Measurement of Anti-Oxidative Capacity and Intracellular ROS by Flow Cytometry

HMCLs cells (1 million cells/1 mL PBS) treated or not treated with 100 μM concentration of H₂O₂ were immediately analyzed for autofluoresnece of FAD (FITC-A channel) and NAD(P)H (UV Blue-A channel) with multicolor flow cytometry (BD LSRFORTESSA). Flowjo histogram normalization was used to overlay untreated versus treated samples.

Post-treatment reactive oxygen species (ROS) levels were determined using the cell-permeable fluorogenic probe DCFDA (Invitrogen Biosciences). Briefly, million cells per 2 mL (2 million cells in 4 mL medium) were cultured overnight. DCFDA (50 μM) was added to suspended cells and incubated for 30 minutes in the dark. Cells were collected and washed once with phosphate-buffered saline (PBS) and split into 2 FACS tubes. One tube contained vehicle (dimethyl sulfoxide), and the other contained lenalidomide (10 μM). Cells were analyzed using a FACSCalibur system (Becton and Dickinson), with excitation and emission spectra set at 488 and 530 nm, respectively. CellQuest software was used to calculate H₂O₂ production by measuring the increase in mean fluorescence.

### Measurement of Total Cellular Antioxidative Capacity

A biochemical test was used to determine the total anti-oxidative capacity of MM cells by evaluating their ability to decompose exogenous H₂O₂ to water and oxygen. Exponentially growing MM cells (sub-cultured for 12 hours) were counted 3 times to ensure that an equal number of cells (1×10⁶ cells) were suspended in equal volumes of PBS. An equal amount (500 μL) of 33% H₂O₂ was added directly to the cells, and the newly formed oxygen bubbles were qualitatively assessed after 15 minutes. This test was sufficiently sensitive to detect differences in cell lines with varying sensitivity to lenalidomide.

### Results

IMIDs Inhibit Peroxidase Mediated Decomposition of H₂O₂ in MM Cells

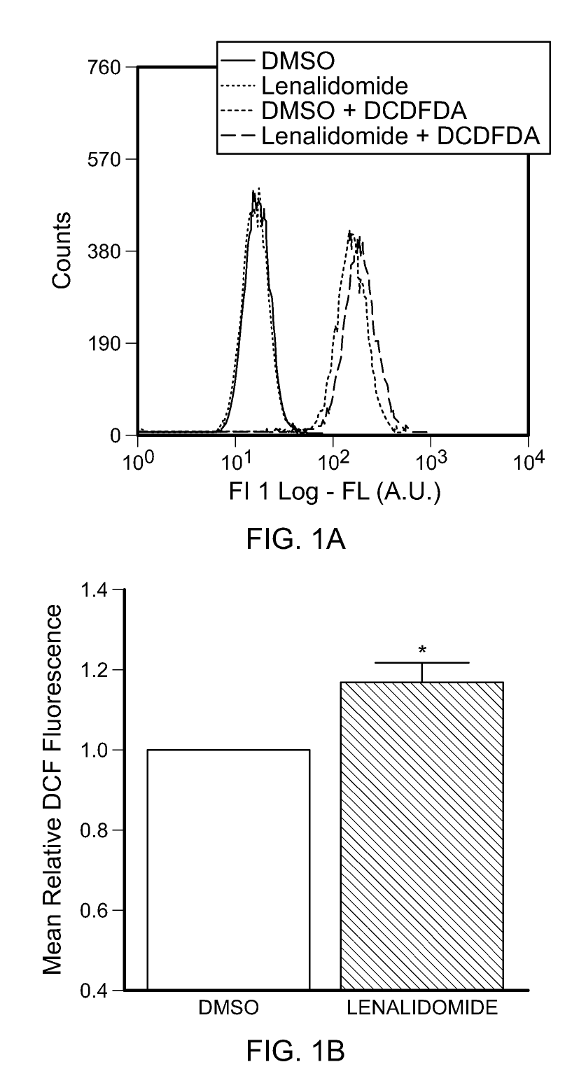

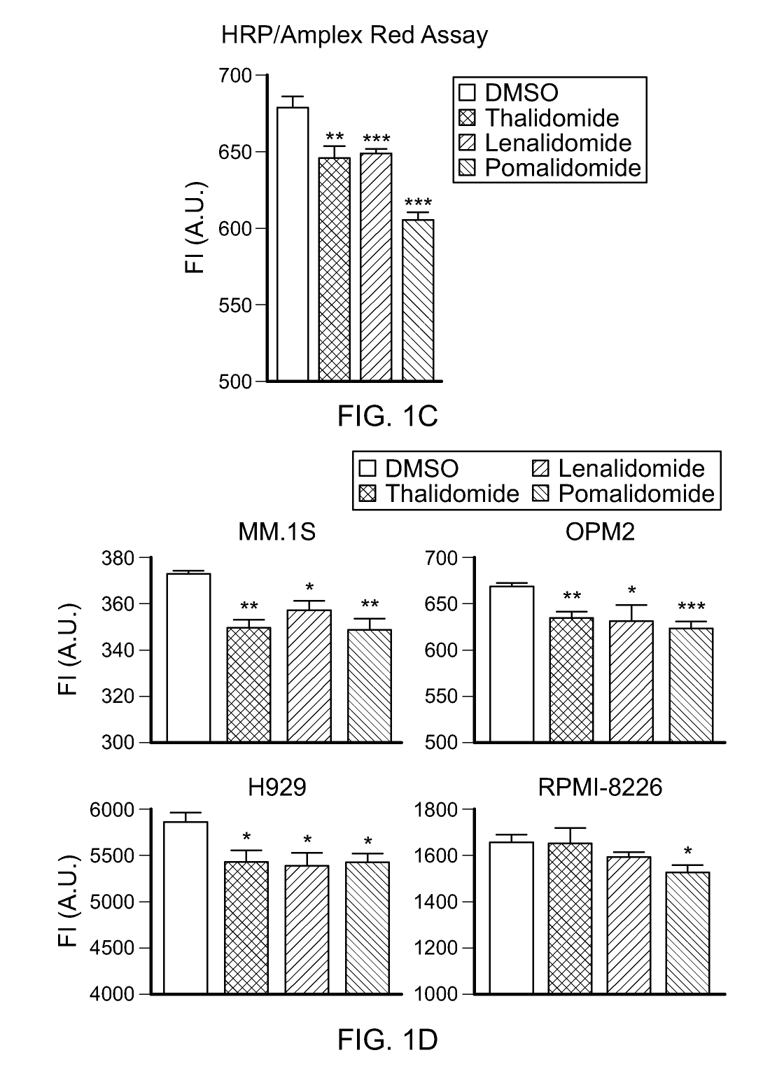

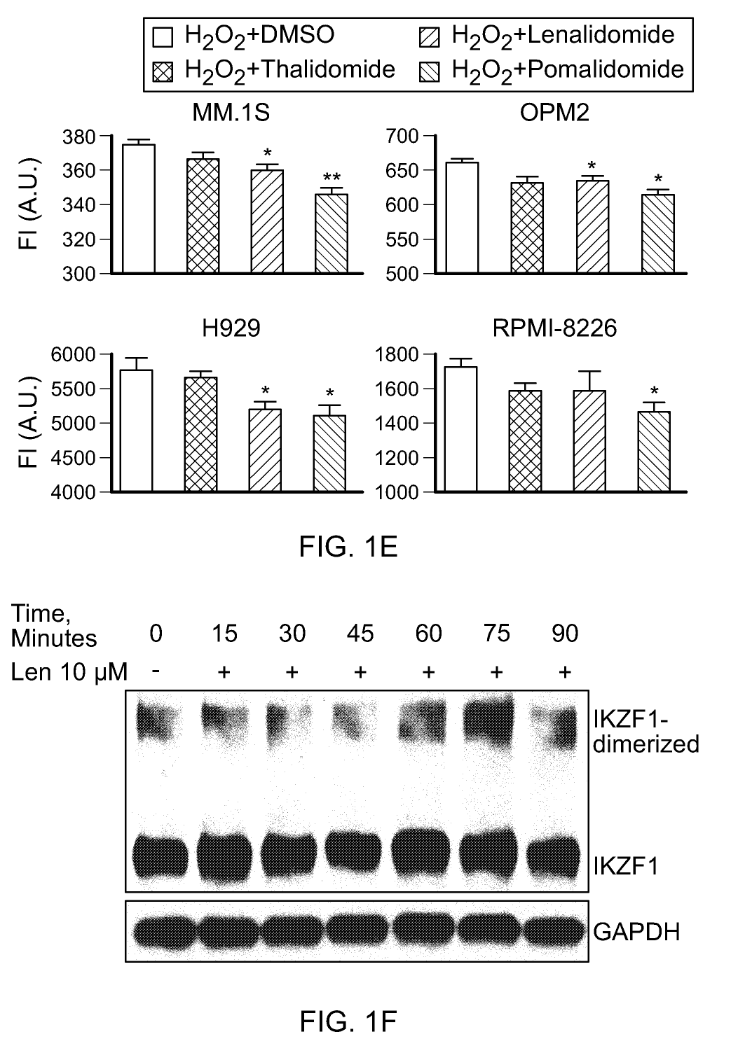

Lenalidomide was tested to determine if it could induce oxidative stress in HMCLs. Cell line MM.1S is highly sensitive to lenalidomide in vitro. Cells were pretreated with 2′,7′-dichlorodihydrofluorescein diacetate (DCFDA) for 30 minutes before exposure to lenalidomide or vehicle control. Lenalidomide-exposed MM.1S cells exhibited increasing intracellular H₂O₂, as evidenced by the fluorescent product 2′,7′-dichlorofluorescein (DCF) detected by fluorescence-activated cell sorting (FACS) analysis (FIG. 1A-B). In the DCF fluorescent assay, the increased signal was evident for only a few minutes after lenalidomide treatment. It later decreased. This raised the question whether lenalidomide is inhibiting peroxidase activity because peroxidase activity is required for DCF fluorescence generation (Dikalov et al., Hypertension. 49(4):717-27 (2007)).

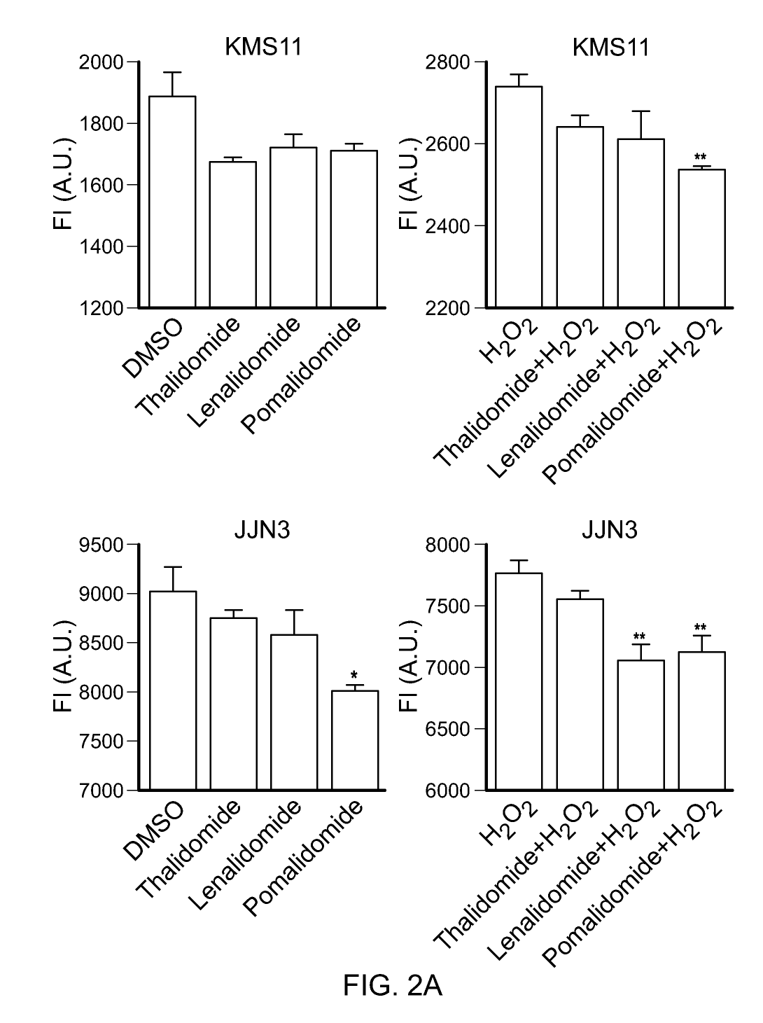

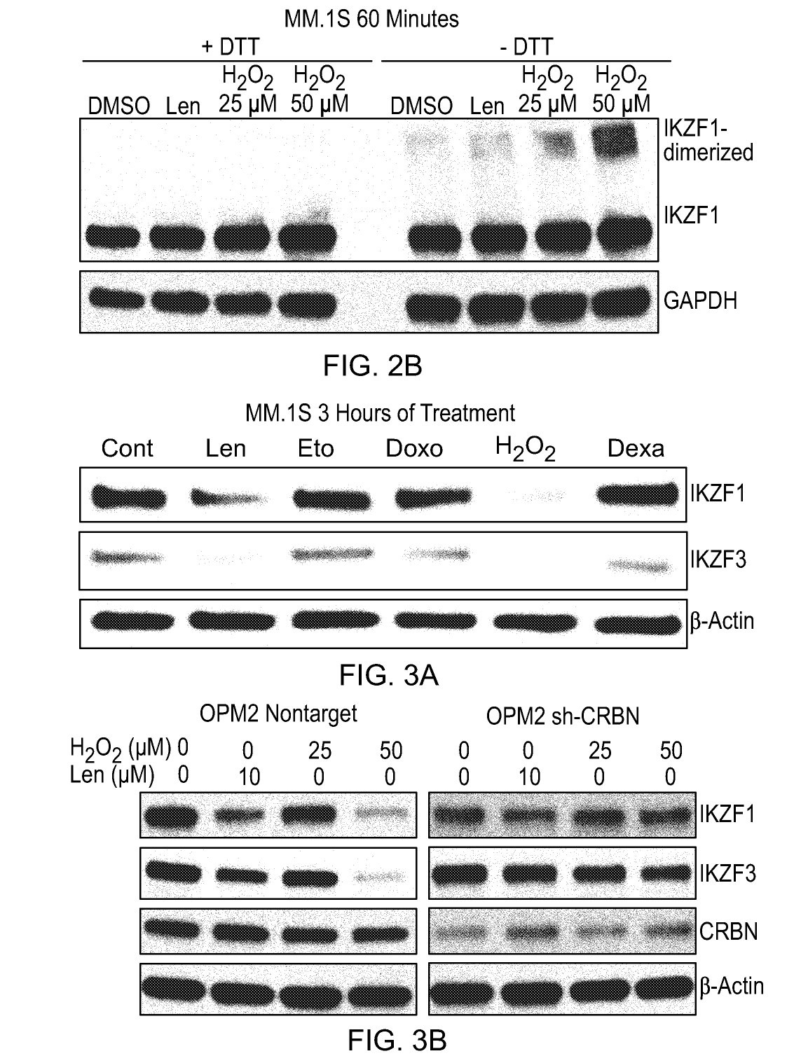

Thalidomide, lenalidomide, and pomalidomide were examined for the ability to inhibit peroxidase activity in vitro using the highly specific peroxidase substrate Amplex Red. In an in vitro assay, decomposition of H₂O₂ by horseradish peroxidase oxidizes Amplex Red to resorufin (oxidized fluorescent product). This was inhibited by immunomodulatory drugs (IMIDs) (thalidomide, lenalidomide, and pomalidomide; FIG. 1C). Next, Amplex Red was used as a substrate for intracellular peroxidase. Thalidomide, lenalidomide, and pomalidomide were found to inhibit intracellular H₂O₂ decomposition mediated by peroxidases in different myeloma cell lines (FIG. 1D and FIG. 2A).

To confirm IMIDs inhibit H₂O₂ decomposition, different HMCLs were treated with external H₂O₂. External H₂O₂ decomposition by cellular peroxidases was inhibited by IMIDs (FIG. 1E). These results demonstrated that thalidomide, lenalidomide, and pomalidomide inhibit intracellular peroxidase-mediated decomposition of H₂O₂. Among different IMIDs analyzed, pomalidomide inhibited intracellular peroxidases more potentially than thalidomide and lenalidomide (FIG. 1C-E and FIG. 2A).

A downstream effect of elevated intracellular H₂O₂ is the induction of protein dimerization by disulfide bonds (Linke et al., Antioxid Redox Signal. 5(4):425-34 (2003); Piwkowska et al., J Cell Physiol. 227(3):1004-16 (2012); Reczek et al., Curr Opin Cell Biol. 33C:8-13 (2014); van der Wijk et al., J Biol Chem. 279(43):44355-61 (2004)). It is hypothesized that intracellular accumulation of H₂O₂ after lenalidomide treatment induces dimerization of proteins such as IKZF1. Indeed, lenalidomide-induced IKZF1 dimerization increased over 75 minutes and later decreased because of protein degradation (FIG. 1F). Similarly, exogenous H₂O₂ (50 μM) also induced IKZF1 dimerization, which peaked at 60 minutes after treatment (FIG. 2B).

H₂O₂ Effectively Degraded IKZF1 and IKZF3 in MM Cells Expressing CRBN

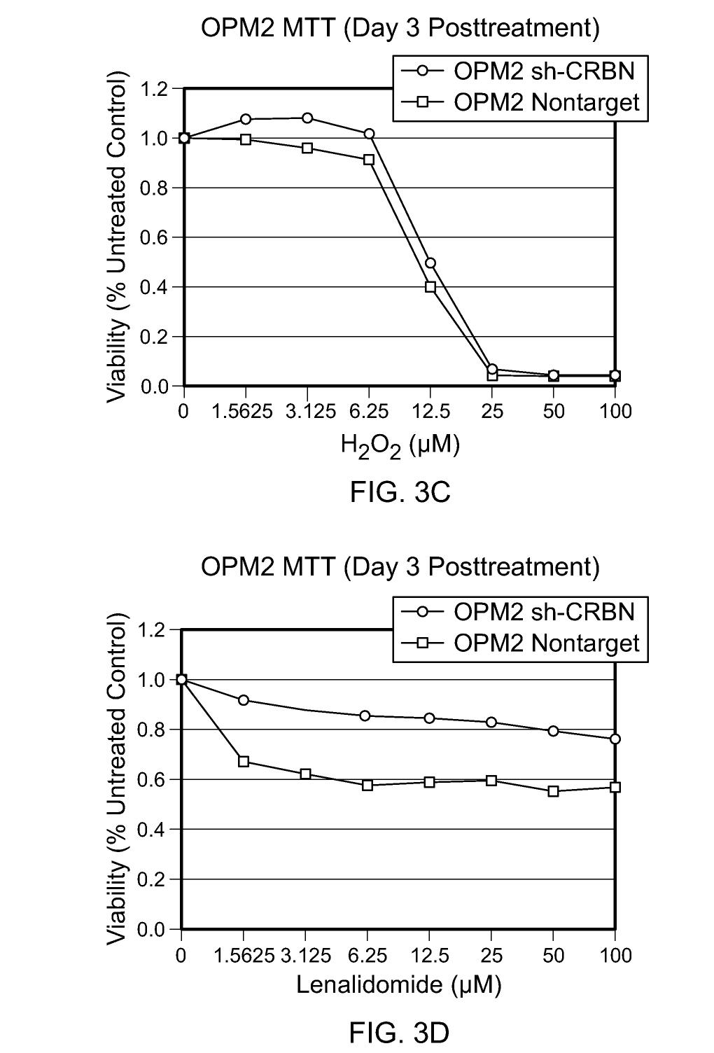

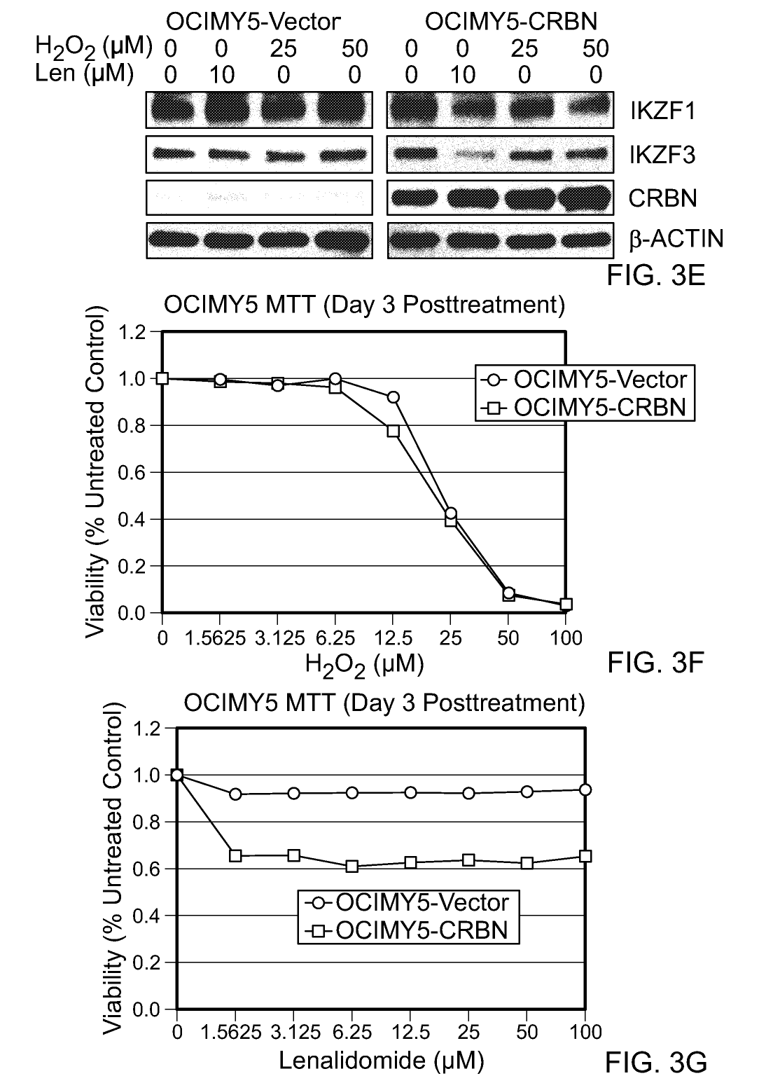

It is believed that lenalidomide-bound CRBN acquires the ability to target IKZF1 and IKZF3 for proteasomal degradation (Fischer et al., Nature. 512(7512):49-53 (2014); Kronke et al., Science. 343(6168):301-5 (2014); Lu et al., Science. 343(6168):305-9 (2014)). After demonstrating that lenalidomide inhibit decomposition of intracellular H₂O₂, it was hypothesized that lenalidomide-induced IKZF1 and IKZF3 degradation was mediated via oxidative stress. MM.1S cells were treated with different drugs that induce oxidative stress. H₂O₂ and lenalidomide degraded IKZF1 and IKZF3 most effectively (degradation was evident within 3 hours; FIG. 3A). These results demonstrate that lenalidomide and exogenous H₂O₂ degraded IKZF1 and IKZF3 by a common mechanism.

To confirm the central role of CRBN in IKZF1 and IKZF3 degradation by H₂O₂-induced oxidative stress, CRBN-knockdown OPM2 isogeneic cells and the CRBN-overexpressing OCIMY-5 cell line (transfected with wild-type CRBN) were examined. OPM2-NT (nontarget short hairpin RNA (shRNA) control) and OPM2-shCRBN (silencing CRBN) cells were treated with lenalidomide (10 μM) and two concentrations of H₂O₂ (25 or 50 μM) for 3 hours. H₂O₂ similarly mediated IKZF1 and IKZF3 degradation in a CRBN-dependent fashion (FIG. 3B). Next, viability of OPM2-NT and OPM2-shCRBN cells with increasing concentrations of lenalidomide and H₂O₂ for 3 days was tested. Lenalidomide-induced cytotoxicity was CRBN dependent, but H₂O₂ was not after 3 days, as shown by 3-(4,5-dimethylthiazol-2-yl)-2,5-diphenyltetrazolium bromide (MTT) assays (FIG. 3C-D).

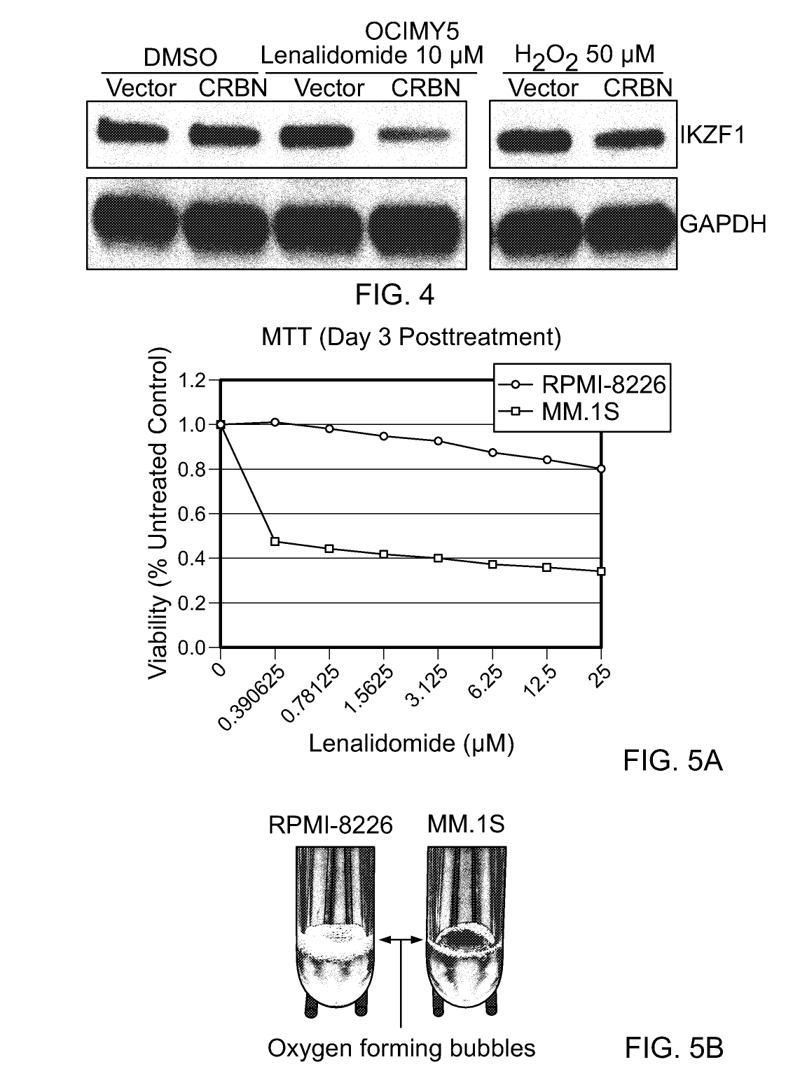

OCIMY-5 cells overexpressing CRBN also exhibited enhanced IKZF1 and IKZF3 degradation within 3 hours of lenalidomide treatment or increasing concentrations of H₂O₂ (FIG. 3E and FIG. 4). Again, although lenalidomide cytotoxicity was CRBN dependent; H₂O₂-mediated cytotoxicity was not, as shown by MTT assay after 3 days of treatment (FIG. 3F-G). Thus, CRBN was required for lenalidomide to induce cytotoxicity and degradation of IKZF1 and IKZF3, but H₂O₂-induced cytotoxicity was independent of CRBN, although CRBN enhanced degradation of IKZF1 and IKZF3. Therefore, CRBN was required for lenalidomide to elevate intracellular H₂O₂, but degradation of IKZF1 and IKZF3 was a consequence of an H₂O₂-mediated, CRBN-dependent proteasomal pathway.

MM Cells with Lower Antioxidative Capacity were More Vulnerable to Lenalidomide-Mediated Cytotoxicity

MM cells with similar levels of CRBN expression can exhibit differential sensitivity to lenalidomide and pomalidomide, suggesting other mechanisms of cytotoxicity. It was hypothesized that the differential capacity to combat H₂O₂ might affect sensitivity to IMIDs. The cellular anti-oxidative capacity as a predictor of lenalidomide sensitivity was analyzed.

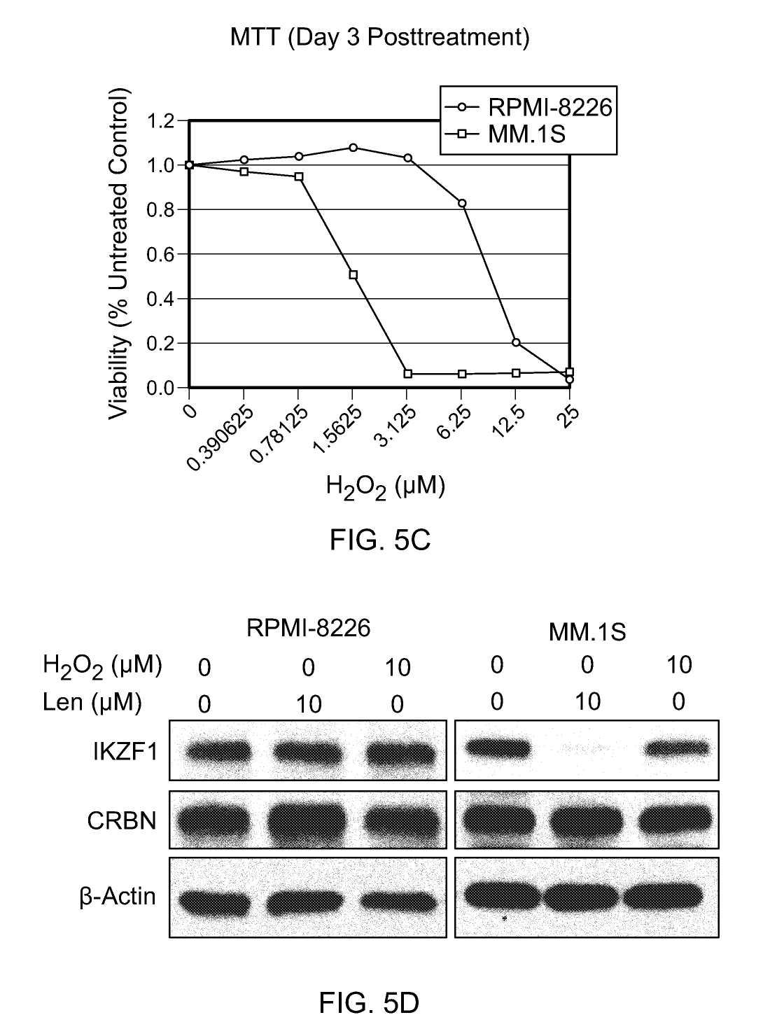

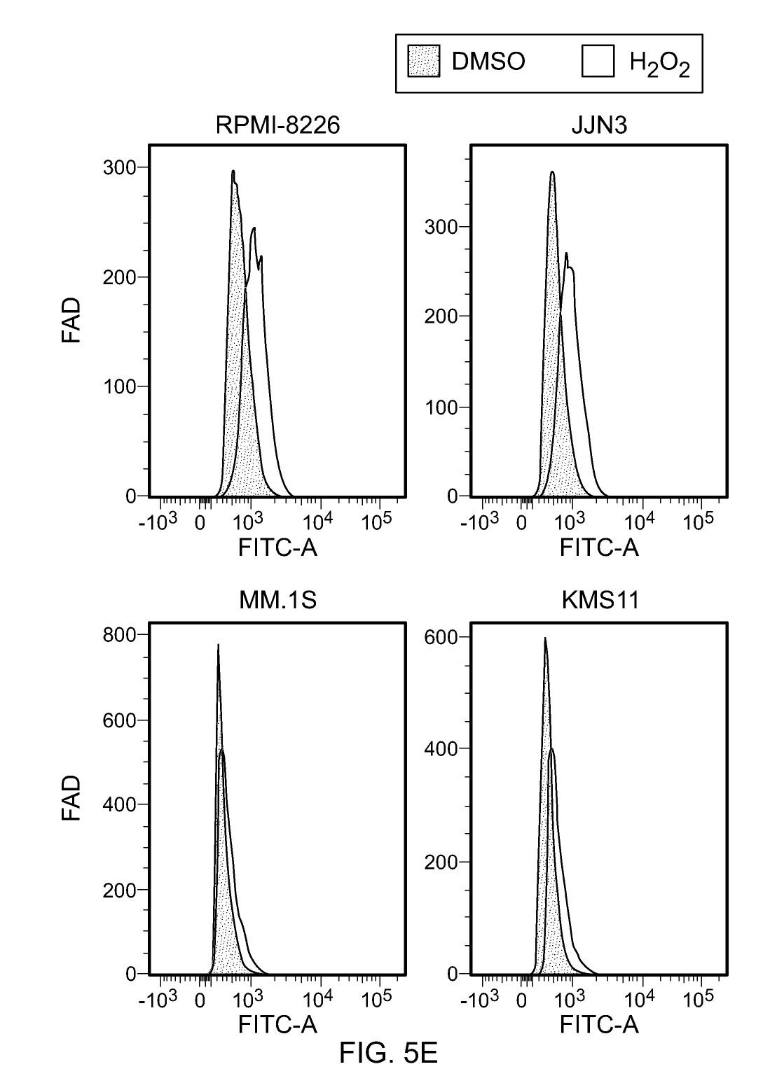

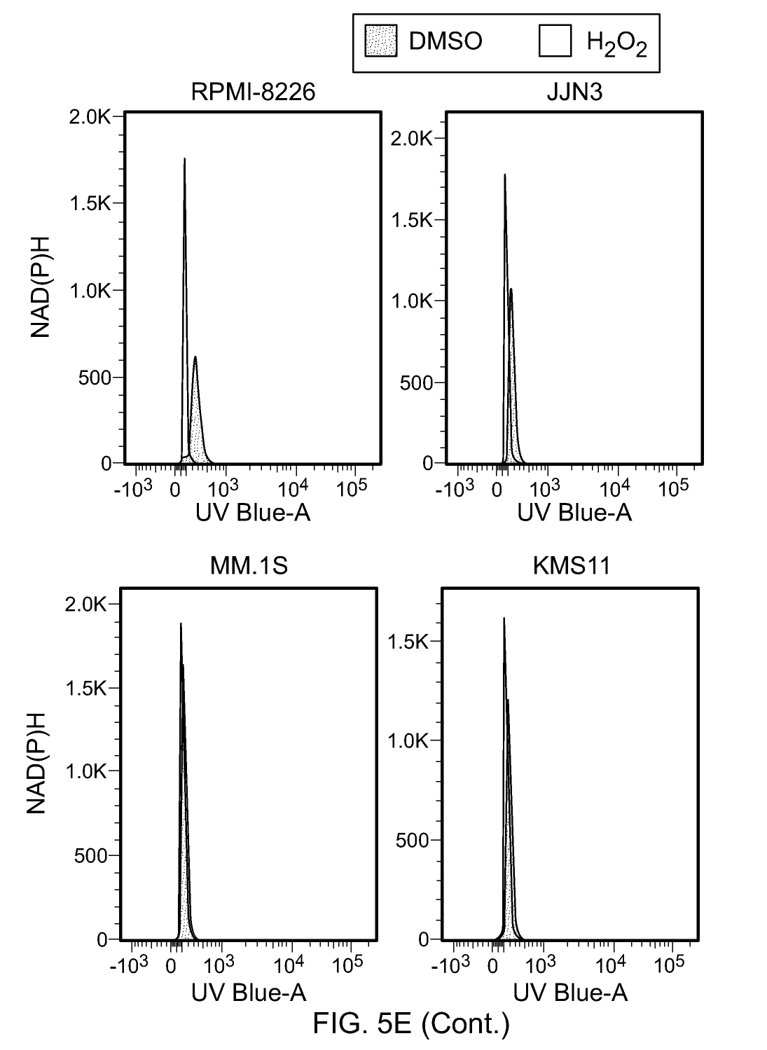

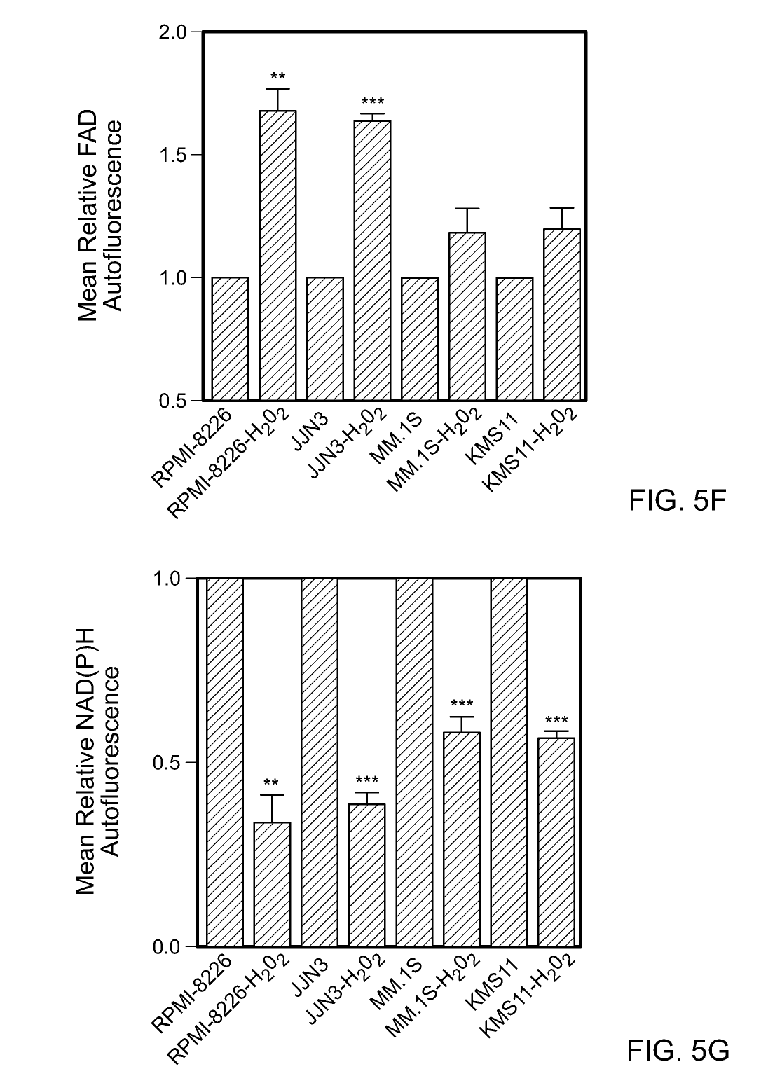

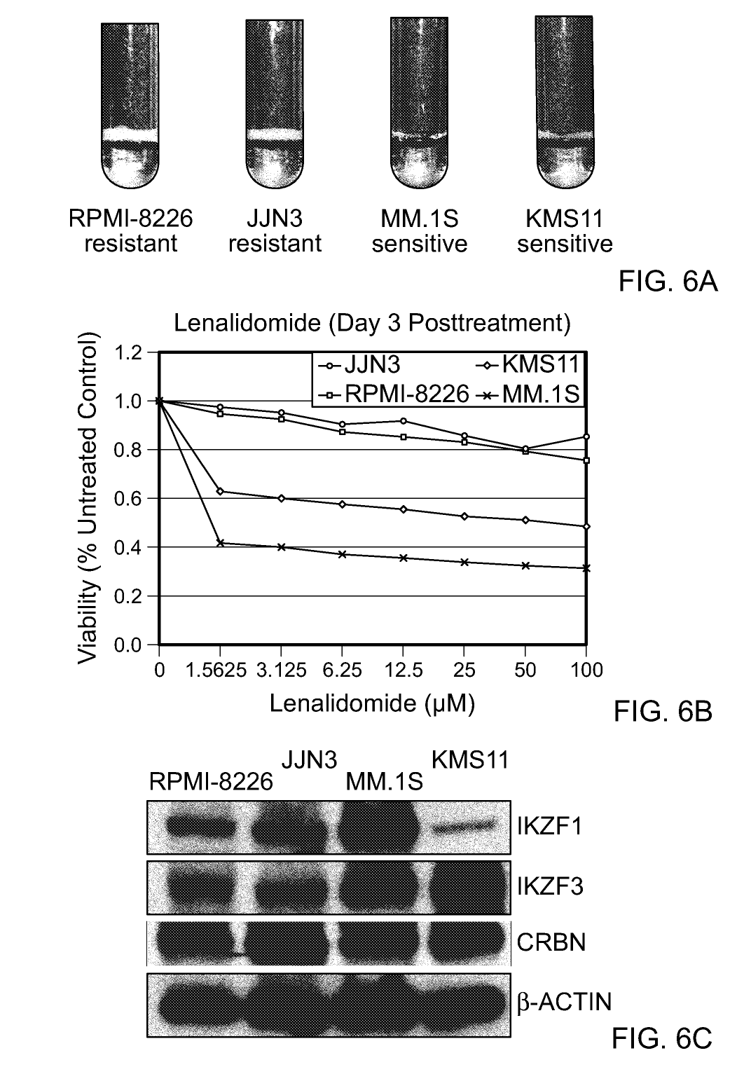

The capacity of MM cells to decompose H₂O₂ was measured via a biochemical test that qualitatively measured the amount of oxygen bubbles formed in vitro after H₂O₂ exposure. MM.1S (hypersensitive to lenalidomide) and RPMI-8226 (resistant to lenalidomide) cell lines were tested (FIG. 5A). An equal number of cells in equal volume were treated with equal amount of H₂O₂. Qualitative examination of the amount of oxygen bubbles formed demonstrated that lenalidomide-resistant MM cells had the greater capacity to decompose H₂O₂ (more bubbles) (FIG. 5B). This differential anti-oxidative capacity appeared to correspond well with external H₂O₂-mediated cytotoxicity. MM.1S was hypersensitive to H₂O₂, and RPMI-8226 was resistant to H₂O₂ (FIG. 5C). After 3 hours of treatment with lenalidomide or H₂O₂, IKZF1 degradation was prominent in MM.1S, but not in RPMI-8226, although both cell lines expressed CRBN protein (FIG. 5D). This observation was reproduced in other CRBN-expressing HMCLs that had differential sensitivity to lenalidomide, including JJN3 (resistant) and KMS11 (sensitive) (FIG. 6).

For the development of more feasible and quantitative assay for determining cellular anti-oxidative capacity, a new strategy was developed to measure total cellular oxidation of FADH₂ and NAD(P)H after H₂O₂ treatment. Cells with a high anti-oxidative capacity generate more oxidized FAD and NAD(P) after H₂O₂ treatment, but cells with a lower anti-oxidative capacity (already under high oxidative state) have less oxidation of FADH₂ and NAD(P)H after H₂O₂ treatment. By taking advantage of auto-fluorescent properties of oxidized FAD and reduced NAD(P)H, H₂O₂ treatment increased oxidized FAD and NAD(P) with increased and decreased autofluoresensce, respectively. In addition, cells with more anti-oxidative capacity and resistance to lenalidomide exhibited a greater increase in FAD autofluoresence and decreased NAD(P)H autofluorescence after 100 μM H₂O₂ treatment (RPMI-8226 and JJN3) than cells with lower anti-oxidative capacity and sensitivity to lenalidomide (MM.1S and KMS11; FIGS. 5E, 5F, and 5G). These results confirm that antioxidant capacity can determine lenalidomide sensitivity among HMCLs with similar CRBN protein expression.

### Lenalidomide-Induced Oxidative Stress Caused Immunoglobulin Light Chain Dimerization and ER Stress

Lenalidomide-induced degradation of IKZF1 and IKZF3 was described elsewhere (Kronke et al., Science. 343(6168):301-5 (2014); Lu et al., Science. 343(6168):305-9 (2014)), but this is not necessarily predictive of cytotoxicity. It was hypothesized that lenalidomide-mediated cytotoxicity in MM is attributable to oxidative damage of intracellular immunoglobulin proteins. Intracellular immunoglobulin light chain (IgL) κ and λ exist in monomeric and dimeric forms (Kaplan et al., Scientific World Journal. 11:726-35 (2011)), and proper folding of IgL is a prerequisite for secretion (Leitzgen et al., J Biol Chem. 272(5):3117-23 (1997); Magrangeas et al., Blood. 103(10):3869-75 (2004)).

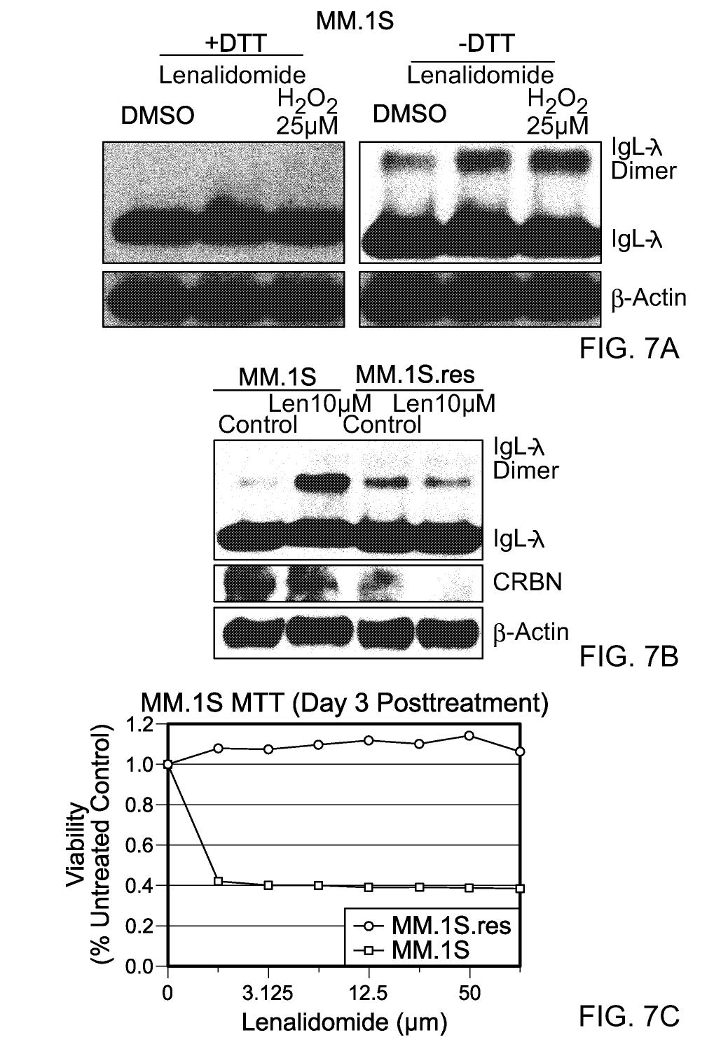

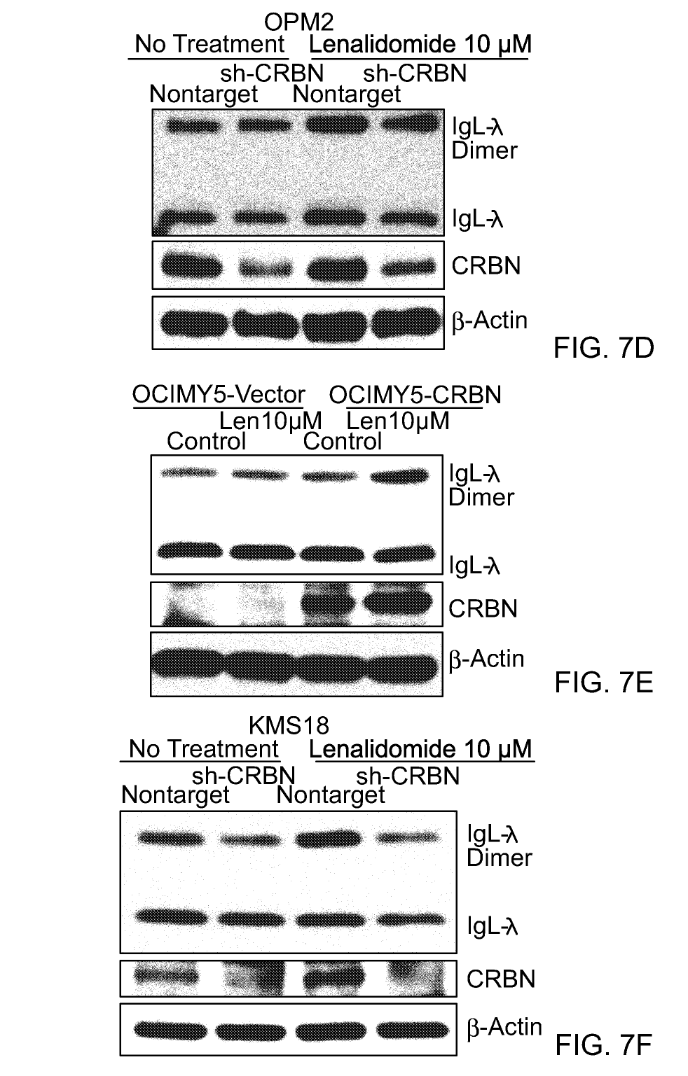

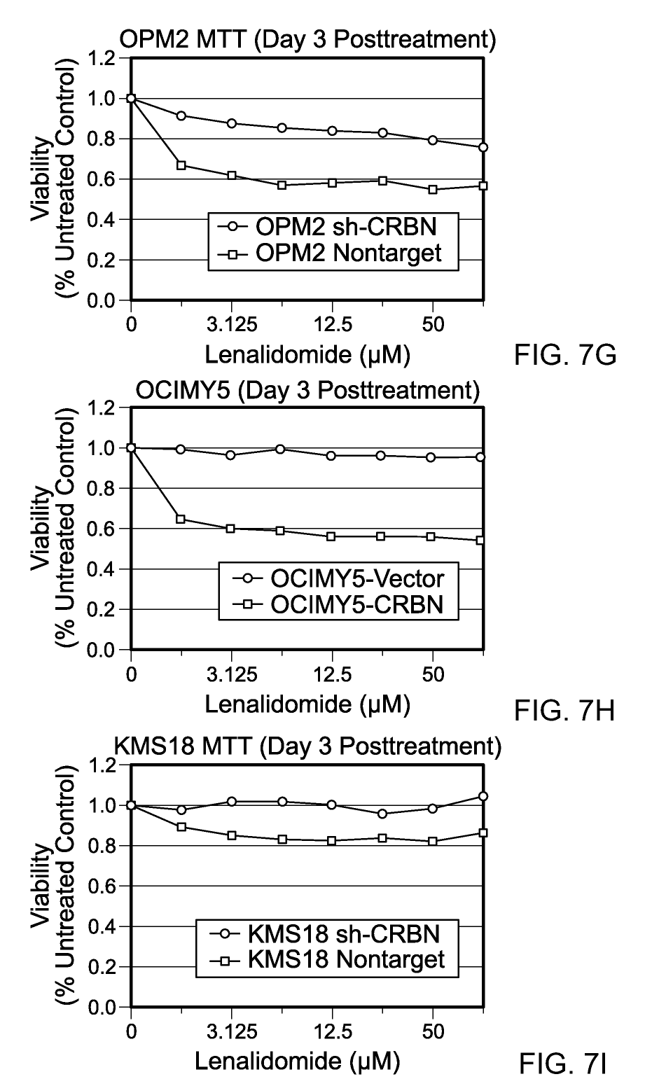

Using MM.1S cells, increased formation of IgL-λ dimers was observed after 3 hours of treatment with lenalidomide or H₂O₂ (FIG. 7A). The increased intermolecular disulfide bond formation further supports lenalidomide-mediated intracellular elevation of H₂O₂. Because the degree of dimerization was somewhat low after 3 hours of lenalidomide treatment, lenalidomide exposure was subsequently prolonged to enhance dimerization in MM.1S. Prolonged treatment was necessary because even though Ikaros degradation occurs rapidly, lenalidomide-induced cytotoxicity is not immediate.

The assay was repeated with lenalidomide-resistant MM.1Sres cells, which were generated by culturing MM.1S in gradually increasing concentrations of lenalidomide (Bjorklund et al., J Biol Chem. 286(13):11009-20 (2011)). CRBN expression in MM.1Sres diminished as lenalidomide resistance increased as described elsewhere (Zhu et al., Blood. 118(18):4771-9 (2011)). MM.1S and MM.1Sres cells were treated with lenalidomide for 3 days, and IgL-λ dimers were observed only in MM.1S (FIG. 7B), which corresponded with lenalidomide sensitivity (FIG. 7C).

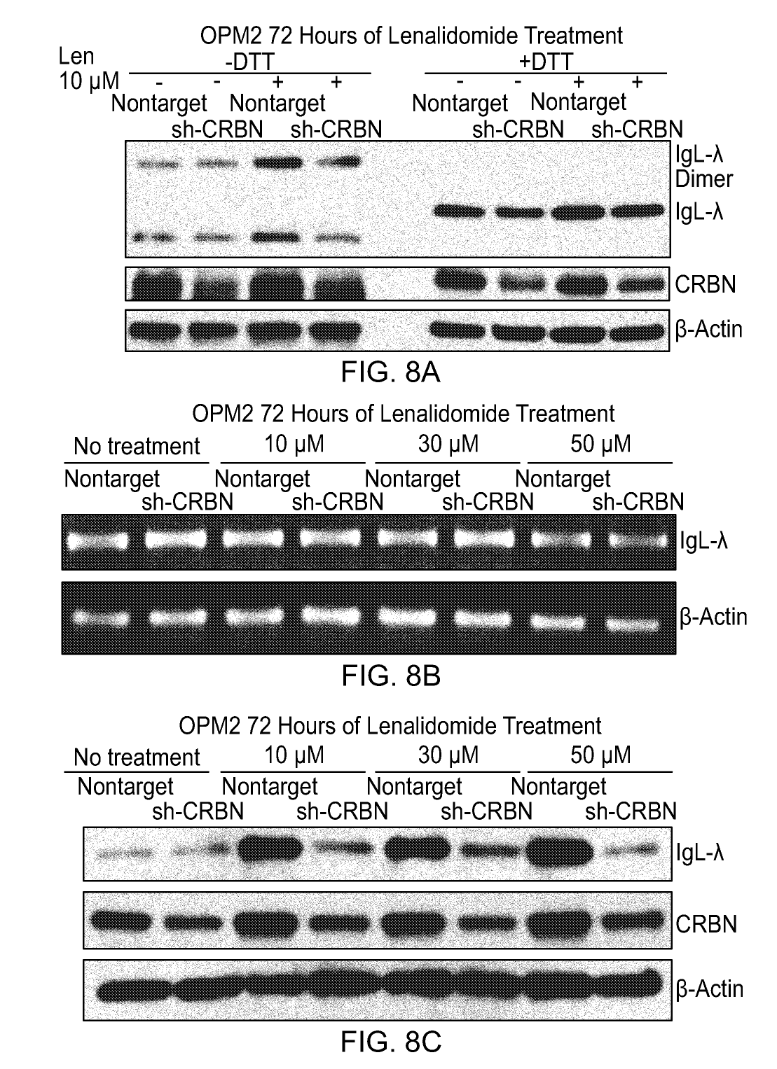

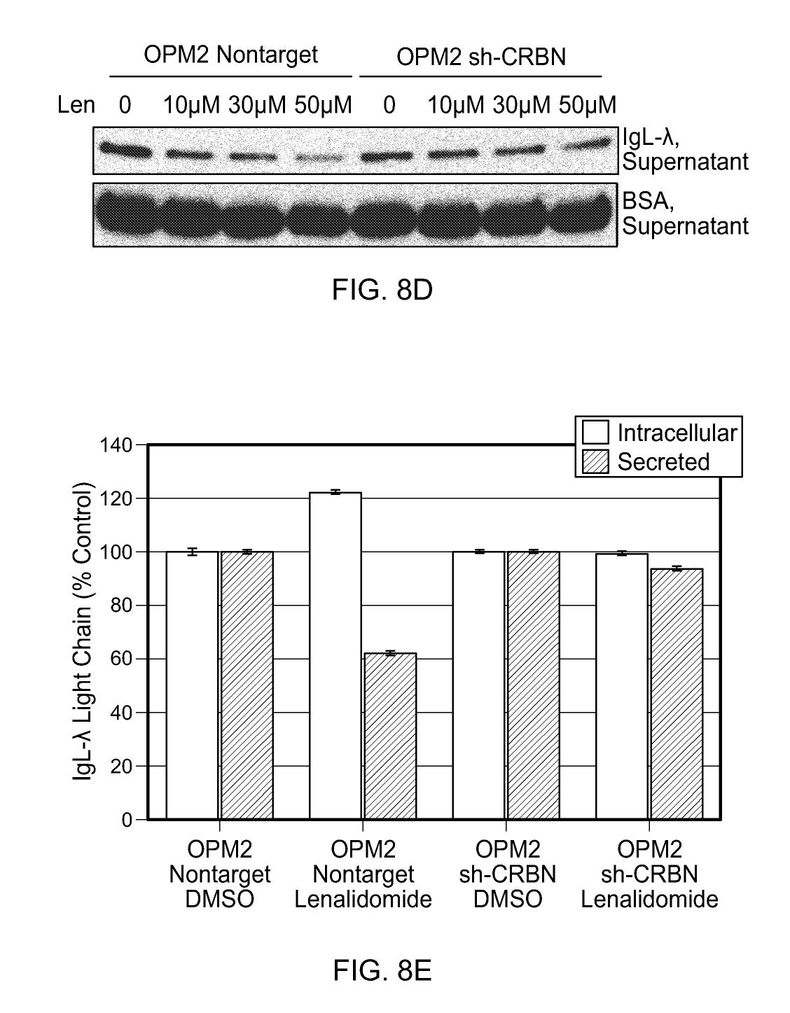

By using other sets of isogenic cells positive and negative for CRBN, it was confirmed that lenalidomide treatment caused accumulation of IgL-λ dimers only in CRBN-positive cells (FIG. 7D-F). Again, this dimerization was completely reduced after electrophoresis with dithiothreitol (FIG. 8A). Dimerization was associated with sensitivity to lenalidomide (FIG. 7G-I). Lenalidomide-induced IgG-λ dimerization lead to decreased secretion and consequent intracellular accumulation of IgG-λ, as evidenced by unchanged IgG-λ mRNA expression (FIG. 8B), increased total intracellular IgL protein (FIG. 8C), and decreased secretion of IgL-λ (FIG. 8D-E).

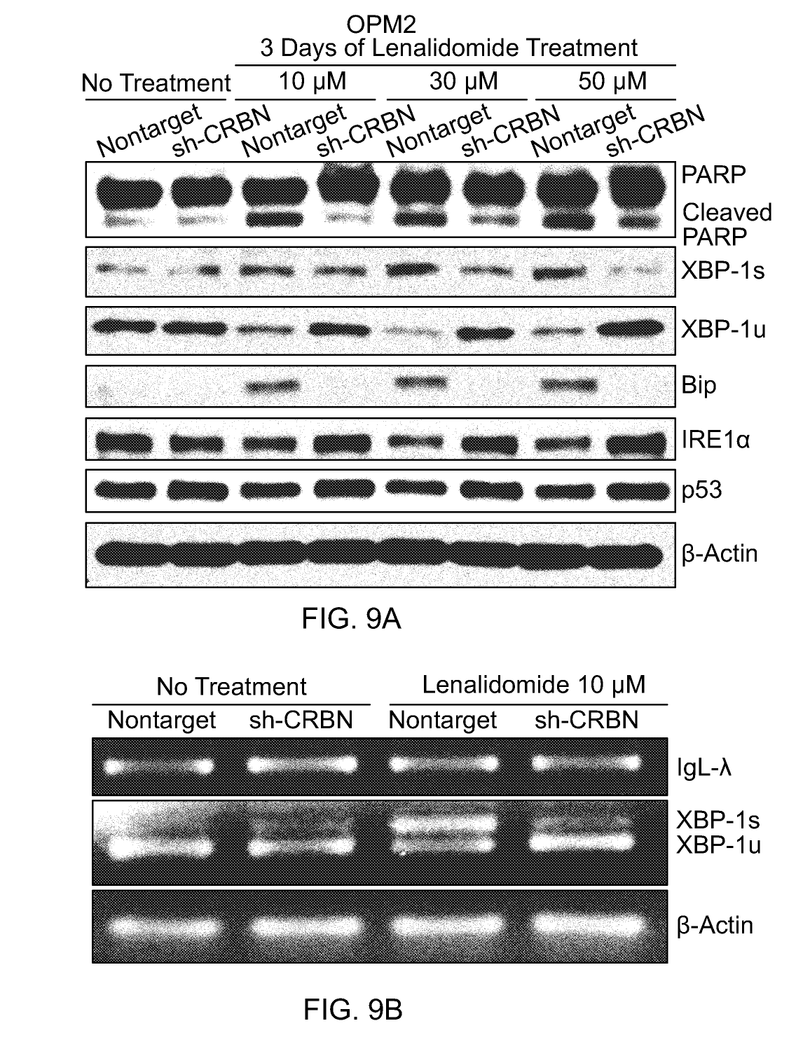

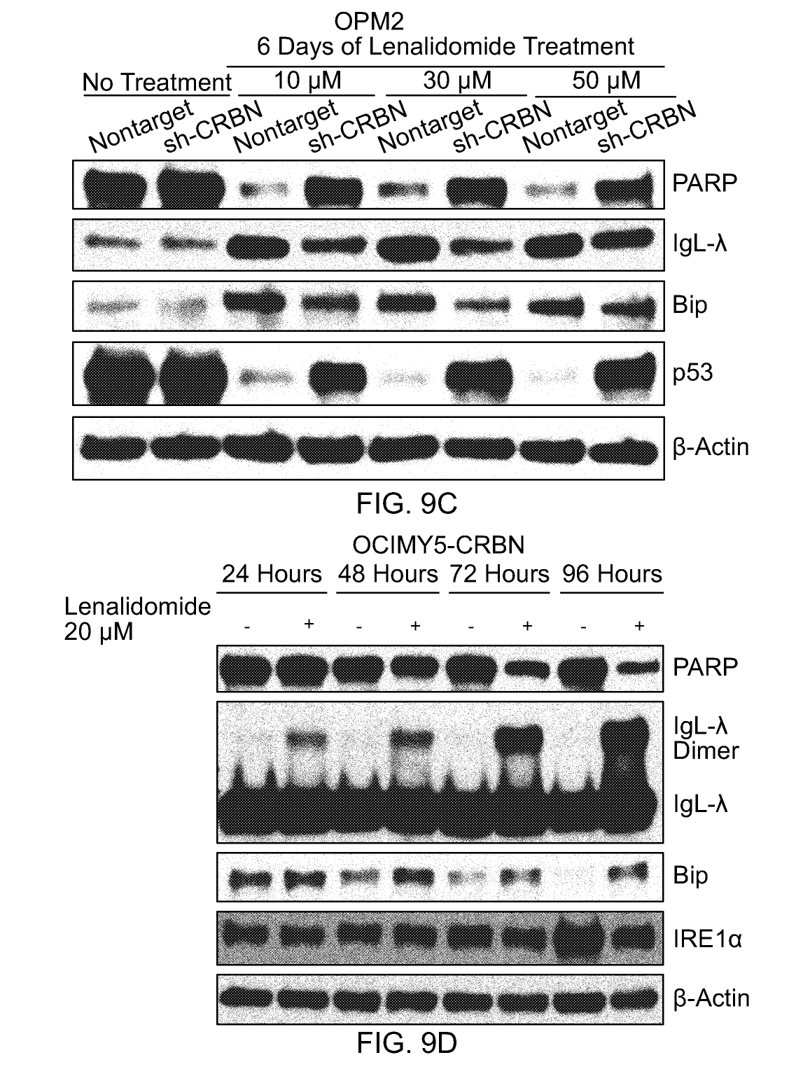

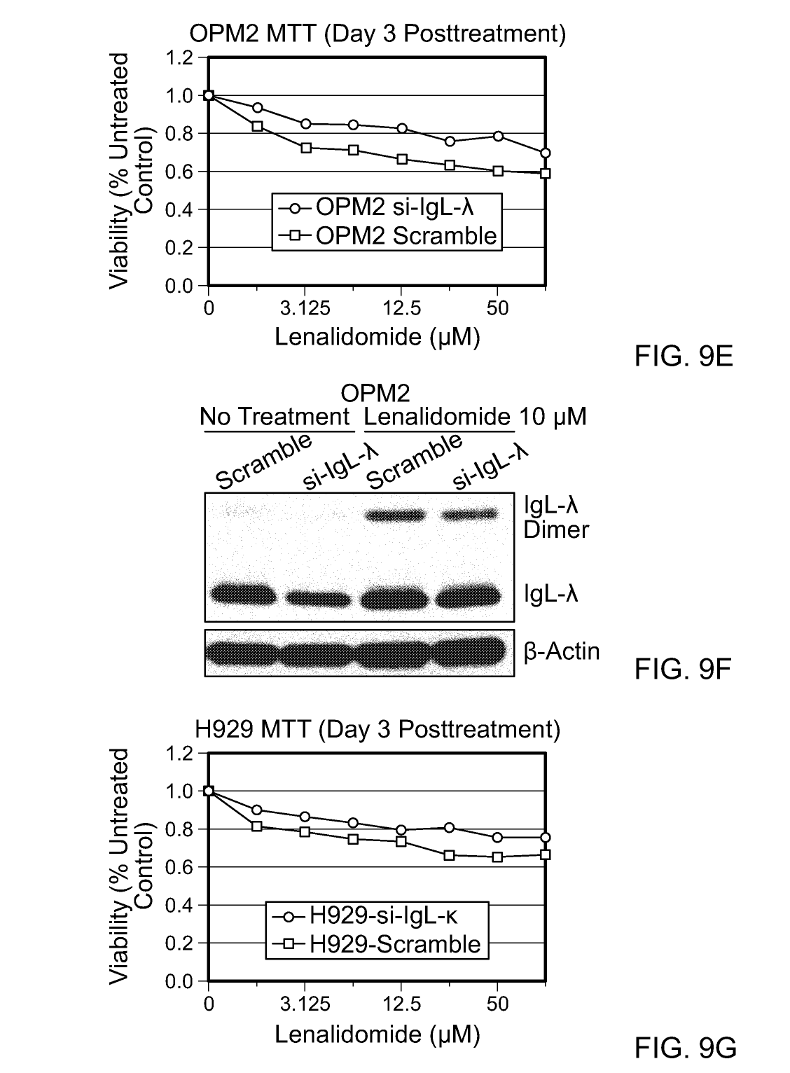

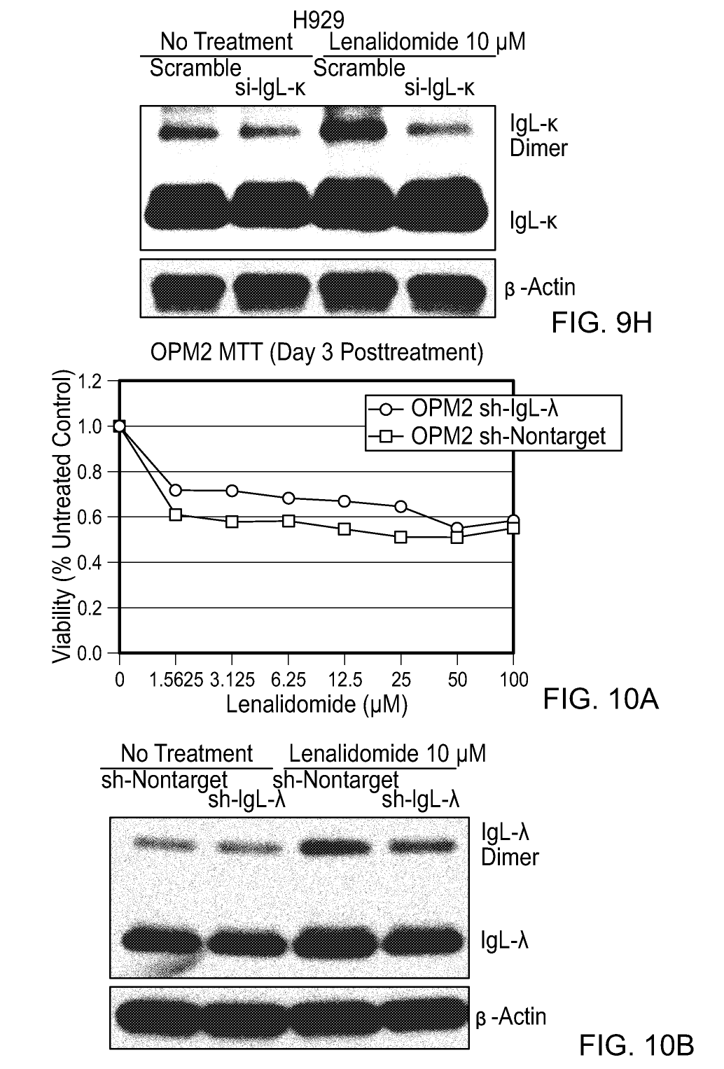

It was postulated that the intracellular accumulation of IgG-λ led to an endoplasmic reticulum (ER) stress response in CRBN-positive cells. After 3 days of treatment with increasing concentrations of lenalidomide, an ER stress response occurred in OPM2-NT cells, but not in CRBN-knockdown cells (FIG. 9A). Immunoblotting revealed decreased XBP-1-unspliced and increased XBP-1-spliced proteins in CRBN-positive (nontarget) cells, but not in CRBN-knockdown cells. These differences were more evident with higher concentrations of lenalidomide (FIG. 9A). Another ER stress marker, GRP78/BiP, accumulated after lenalidomide treatment in CRBN-positive cells (FIG. 9A). To further confirm that lenalidomide induced ER stress, mRNA splicing of XBP-1 after lenalidomide treatment was analyzed. Splicing was more evident in OPM2-NT cells than CRBN-knockdown cells (FIG. 9B) after 3 days of lenalidomide treatment. The inositol-requiring enzyme 1 (IRE1α) protein level markedly decreased in CRBN-positive cells after lenalidomide treatment (FIG. 9A), indicating elevated IRE1α endoribonuclease activity during ER stress (Tirasophon et al., Genes Dev. 14(21):2725-36 (2000)). In addition, p53 protein levels decreased after lenalidomide treatment in CRBN-positive cells. Inactivation of the tumor suppressor p53 by degradation is a mechanism cells use to adapt to ER stress (Pluquet et al., Mol Cell Biol. 25(21):9392-405 (2005)). Minimal apoptosis was detected in OPM2-NT cells, as shown by low levels of poly (ADP-ribose) polymerase (PARP) cleavage and p53 degradation (FIG. 9A) after 3 days of drug treatment. Therefore, OPM2-NT and OPM2-shCRBN cells were treated with lenalidomide for 6 days, and the p53 and PARP levels were analyzed. Immunoblots revealed complete degradation of p53 and PARP proteins (cleaved PARP was not detected because of complete degradation at day 6) in CRBN-positive lenalidomide-sensitive cells (FIG. 9C). Moreover, to better understand lenalidomide-mediated IgL dimerization and accumulation as a mediator of time-dependent progressive ER stress, the immunoblots were repeated using CRBN-overexpressing OCIMY5 cells (the OCIMY5 parent cell line was not responsive to lenalidomide (FIGS. 7E and 7H)). OCIMY5 cells stably transfected with CRBN were treated with lenalidomide and exhibited progressive IgL-λ dimer accumulation, ER stress, and total PARP degradation, signs of later stage apoptosis (FIG. 9D).

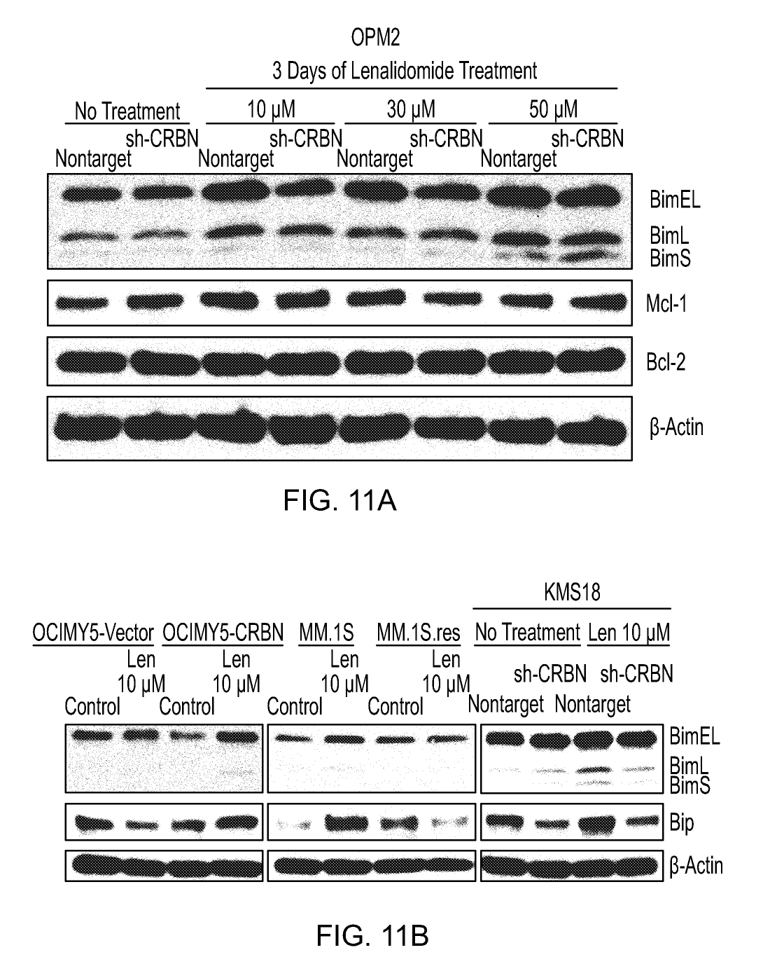

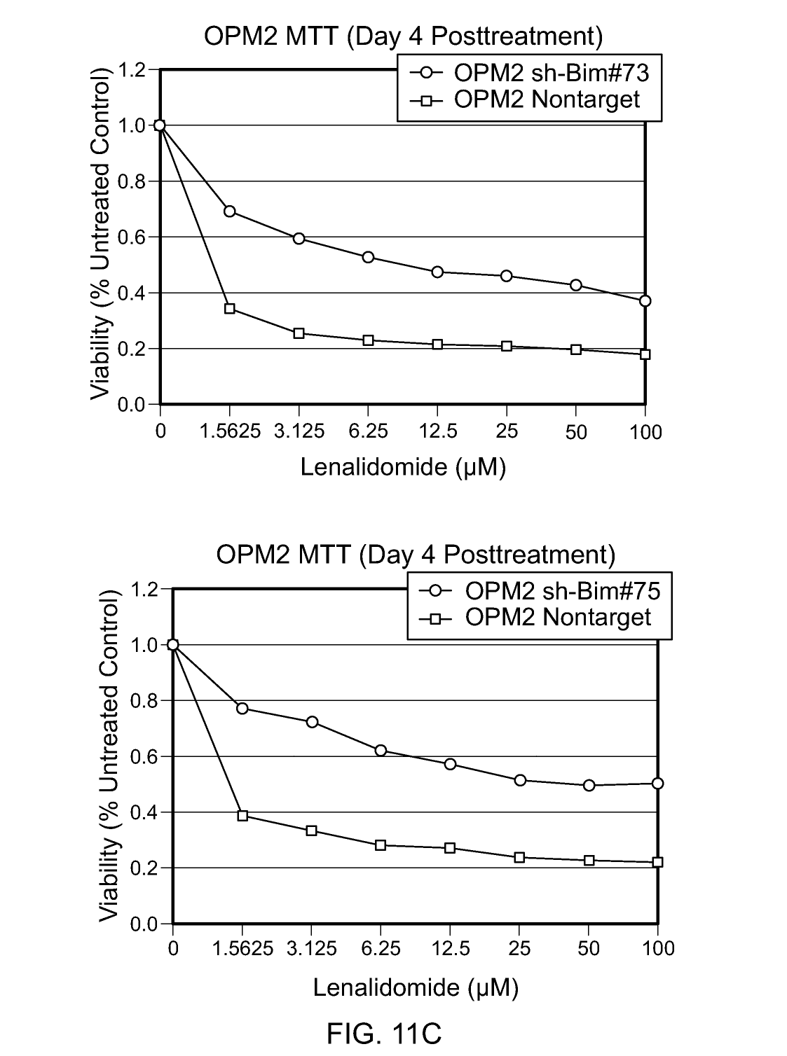

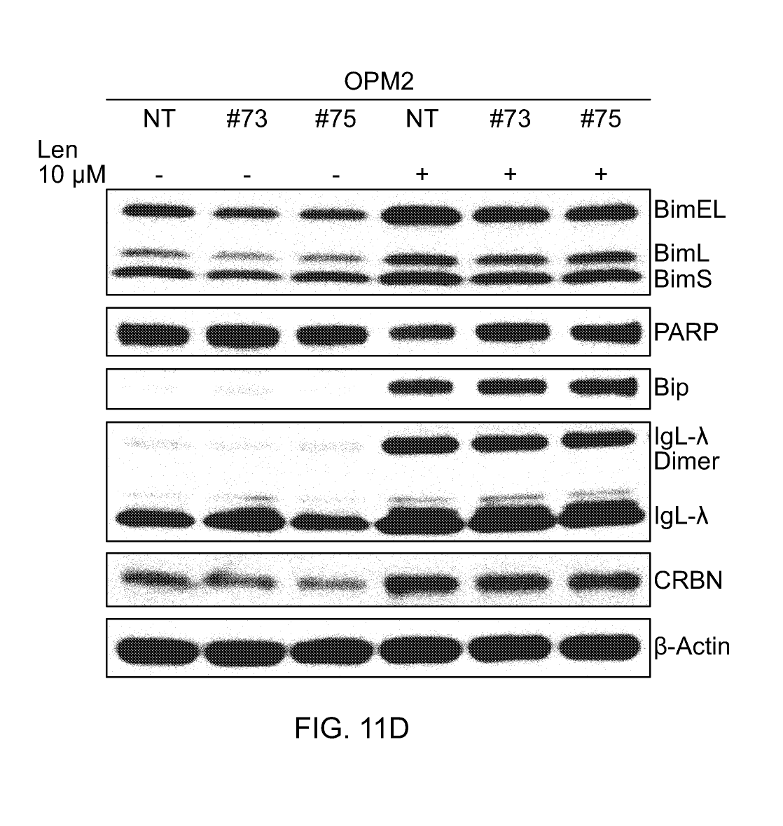

Other isogenic HMCLs MM.1S (lenalidomide sensitive) and MM.1Sres (lenalidomide resistant), as well as KMS18-NT and KMS18-shCRBN, were analyzed. Lenalidomide induced ER stress-mediated accumulation of Bip protein in CRBN-positive cells, but not in CRBN-negative cells (FIG. 11B). To further confirm that lenalidomide-mediated ER stress in CRBN-expressing cells is due to accumulation of IgL, IgL-λ was knocked down in OPM2 cells with piLenti-siRNA (FIG. 9E-F). Only about 40% of IgL-λ production was knocked down in OPM2 (because of its high levels of expression). The remaining expression was much higher than that of (3-actin, the housekeeping gene. Nevertheless, this limited knockdown of IgL-λ in OPM2 cells reduced lenalidomide sensitivity (FIG. 9E) and decreased IgL-λ dimerization after 3 days of lenalidomide treatment compared with control cells (FIG. 9F).

Another IgL-λ knockdown clone was generated by using the stable shRNA method. This clone also had lenalidomide resistance compared with controls (FIG. 10). Moreover, knockdown of IgL mediated resistance to lenalidomide was not restricted to IgL-λ expressing cell lines because the limited knockdown of IgL-κ in H929 likewise reduced lenalidomide sensitivity and exhibited IgL-κ dimerization (FIG. 9G-H).

### Lenalidomide-Induced Oxidative Stress Triggers Cytotoxicity by Activating BH3 Protein Bim in MM

Bim activation induced apoptosis after lenalidomide treatment in CRBN-positive MM cells. CRBN-expressing and CRBN-knockdown OPM2 cells were treated with lenalidomide for 3 days, and cell lysates were immunoblotted and probed for various proapoptotic and antiapoptotic proteins. BH3-only protein Bim was activated after lenalidomide-induced ER stress (FIG. 11A). Bim has 3 isoforms, BimS, BimL, and BimEL. Their differing proapoptotic potencies are partly due to differences in their interactions with the dynein motor complex (Faber et al., Adv Pharmacol. 65:519-42 (2012)).

Accumulation of Bim was observed, especially BimEL, after lenalidomide treatment in CRBN-positive, lenalidomide-sensitive cells. Mcl1 and Bcl2 antiapoptotic proteins did not change markedly after lenalidomide treatment (FIG. 11A). Other CRBN-negative and CRBN-positive isogenic cell lines were analyzed, and Bim activation and ER-stress (Bip accumulation is a sign of ER-stress) were confirmed after lenalidomide treatment (FIG. 11B). Protein levels of all 3 Bim isoforms, particularly BimEL, markedly increased after lenalidomide treatment in CRBN-positive isogenic MM cell lines.

To confirm Bim involvement in lenalidomide-induced apoptosis, stable shRNA expression was used to knock down Bim in OPM2 cells. Two different OPM2 clones (#73 and #75) with downregulated Bim were established and treated with lenalidomide. Because lenalidomide induced late apoptosis in OPM2 cells, a day-4 MTT assay for cell viability was performed. Both Bim knockdown clones were less sensitive to lenalidomide than control cells (FIG. 11C). Moreover, Bim knockdown cells underwent less PARP cleavage compared with control cells (FIG. 11D). Bim knockdown did not inhibit IgL-λ dimerization and accumulation or subsequent ER stress after lenalidomide treatment. Bim knockdown cells also exhibited elevated Bip protein accumulation (a marker of ER stress), like nontarget (control) cells after lenalidomide treatment, but ER stress-mediated apoptosis was diminished in Bim knockdown clones (FIG. 11D). Thus, BimEL appeared to be a downstream effector of lenalidomide-induced, ER stress-mediated apoptosis in MM.

Pretreatment with Lenalidomide Enhanced Bortezomib Sensitivity in MM

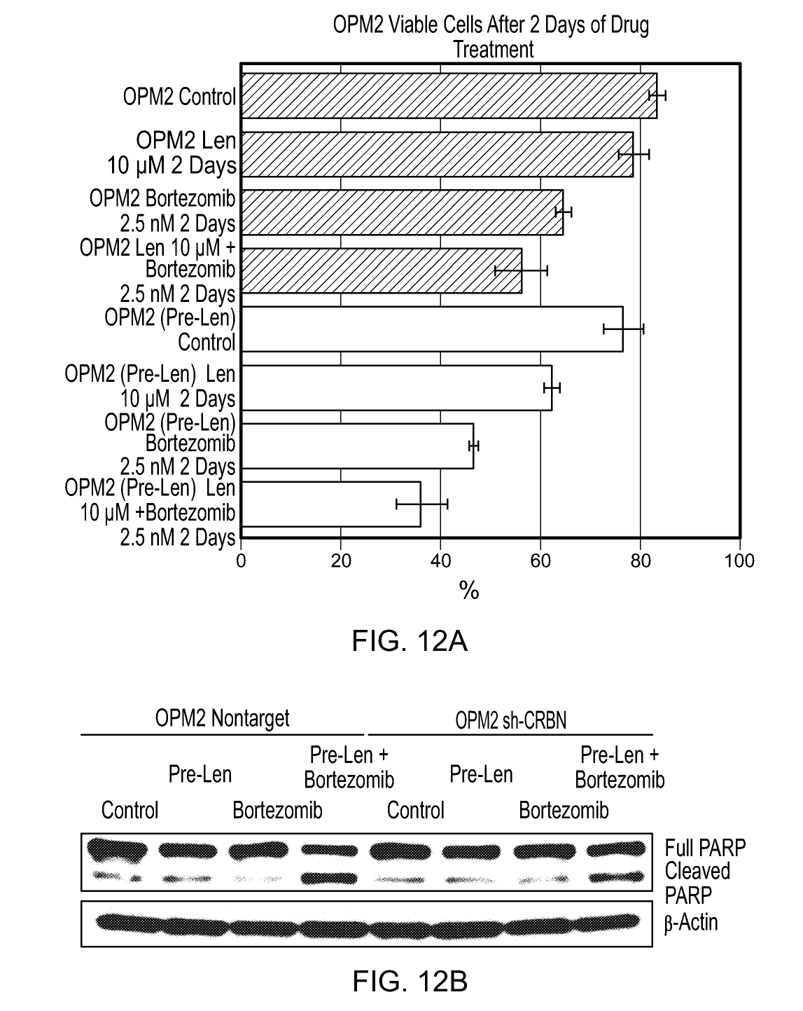

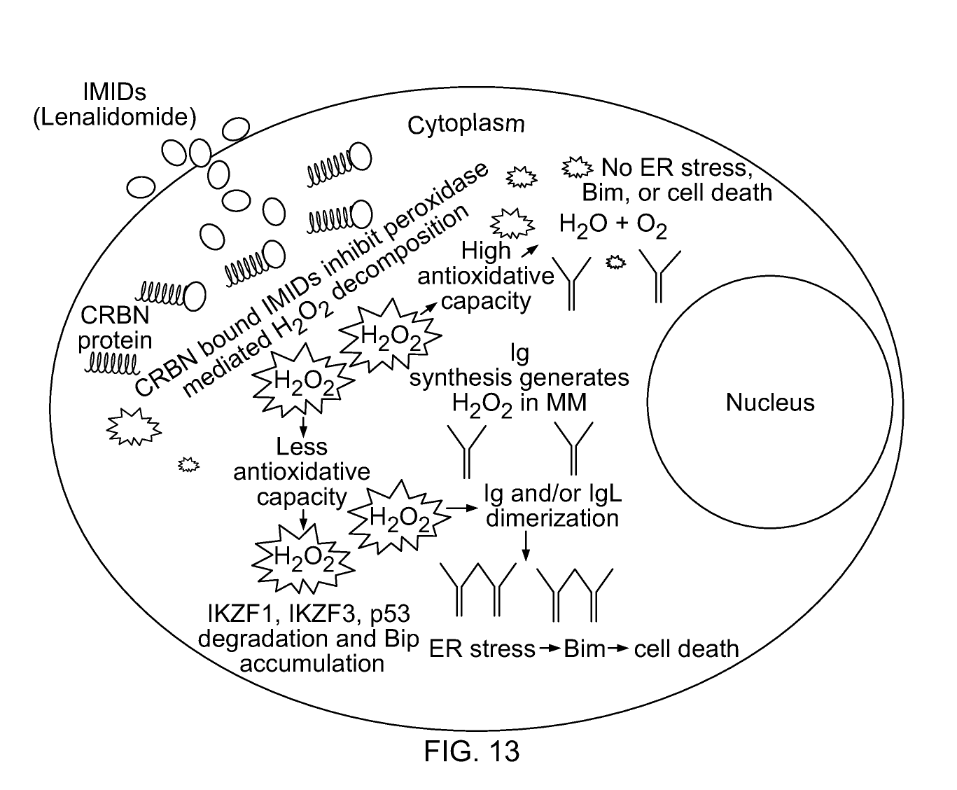

From the above, it was postulated that lenalidomide-mediated ER stress would positively enhance bortezomib-mediated cytotoxicity in MM. To translate these findings to clinical applications, MM cells were pretreated with lenalidomide and then treated with bortezomib. OPM2 cells pretreated with lenalidomide for two days clearly exhibited increased sensitivity to bortezomib-induced apoptosis compared with cells that were not pretreated (FIG. 12A). Apoptosis of OPM2 cells with or without lenalidomide pretreatment, followed by treatment with bortezomib alone, lenalidomide alone, or bortezomib plus lenalidomide were also examined. The combination of bortezomib plus lenalidomide induced more apoptosis, and this effect was significantly enhanced in lenalidomide-pretreated cells (FIG. 12A). Next, whether CRBN-positive cells were more prone to apoptosis after lenalidomide pretreatment followed by bortezomib treatment was analyzed. OPM2-NT and OPM2-shCRBN cells were pretreated with lenalidomide for 2 days, and the cells were washed and then treated with bortezomib for an additional 2 days. Lenalidomide pretreatment enhanced bortezomib-induced PARP cleavage in OPM2 CRBN-positive cells, but cleavage was reduced in CRBN knockdown cells (FIG. 12B). These results demonstrate that one could use serial therapy with lenalidomide pretreatment (even for a few days) followed by bortezomib or other proteasome inhibitors such as carfilzomib in combination with lenalidomide.

The results provided herein demonstrate that IMIDs inhibit peroxidase mediated H₂O₂ decomposition in MM cells, that H₂O₂ induces degradation of IKZF1 and IKZF3 in cereblon-positive cells, that cellular antioxidative capacity determines sensitivity to lenalidomide, and that elevated H₂O₂ mediates immunoglobulin dimerization and intracellular stress.

### Additional Results

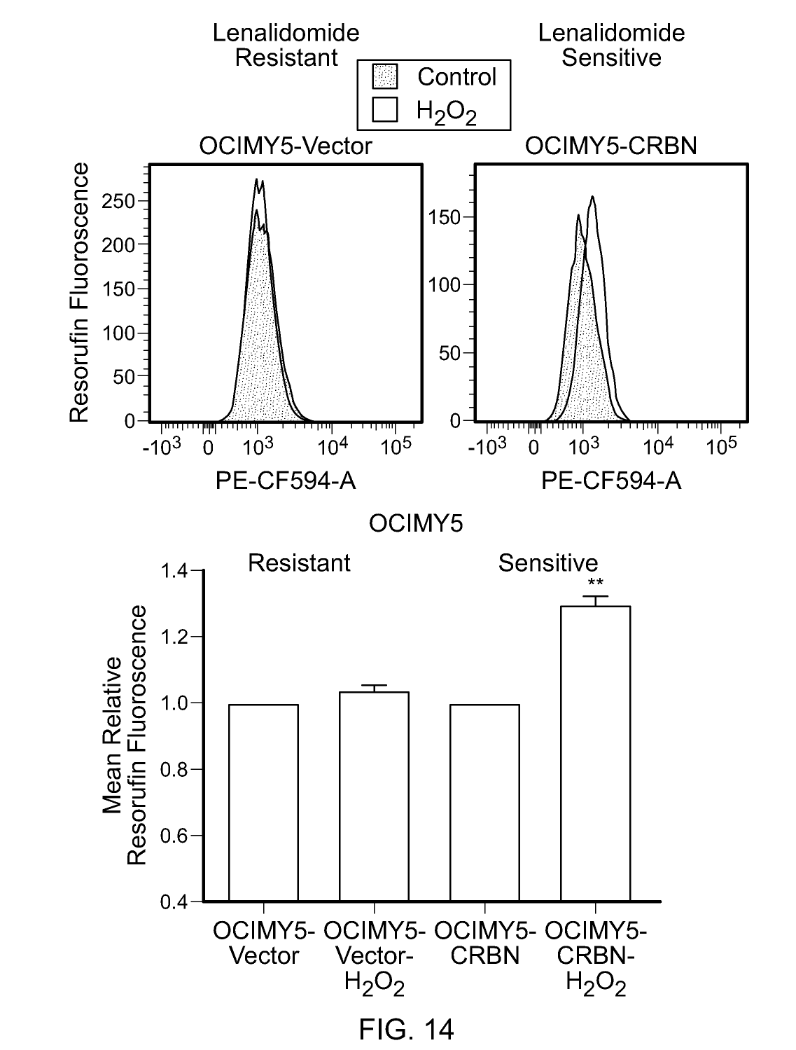

In another test, treatment with amplex red (a fluorescent substrate for peroxidases) and exogenous H₂O₂ treatment was used to detect the anti-oxidative capacity of cancer cells (FIG. 14). If cells are taking more amplex red and converting it to resorufin after external H₂O₂ treatment, then those cells were identified as having less anti-oxidative capacity and were identified as being sensitive to IMIDs (e.g., lenalidomide) (FIG. 14).

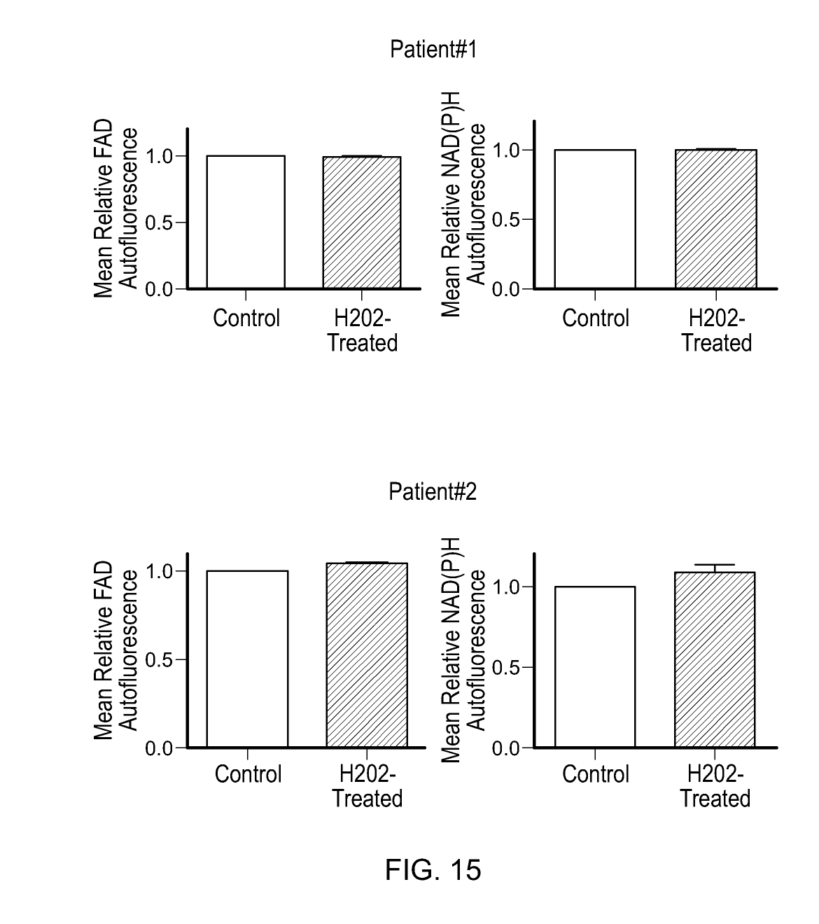

In another study, cancer patients (e.g., myeloma patients) were identified as being sensitive to IMIDs by assessing oxidation of NAD(P)H and FADH₂. Briefly, an anti-oxidative capacity assay was used to identify two patients as being sensitive to IMIDs (lenalidomide) because after H₂O₂ treatment, no further increase in oxidation of NAD(P)H and FADH₂ was observed (FIG. 15).

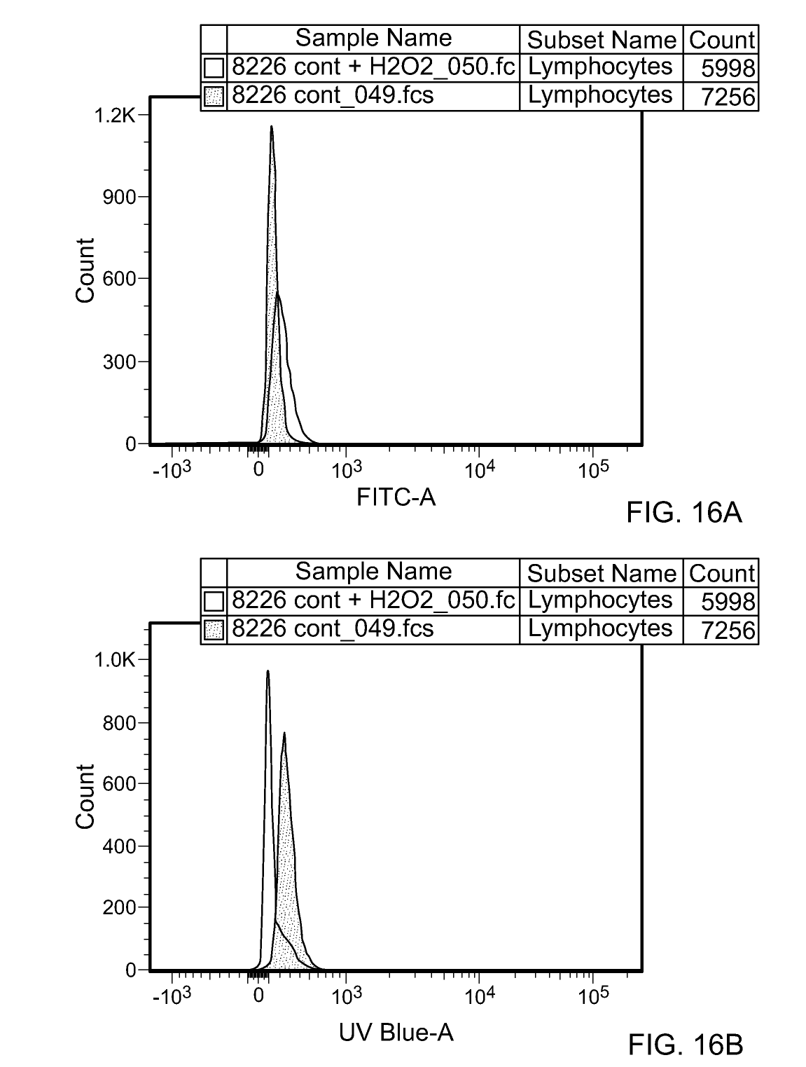

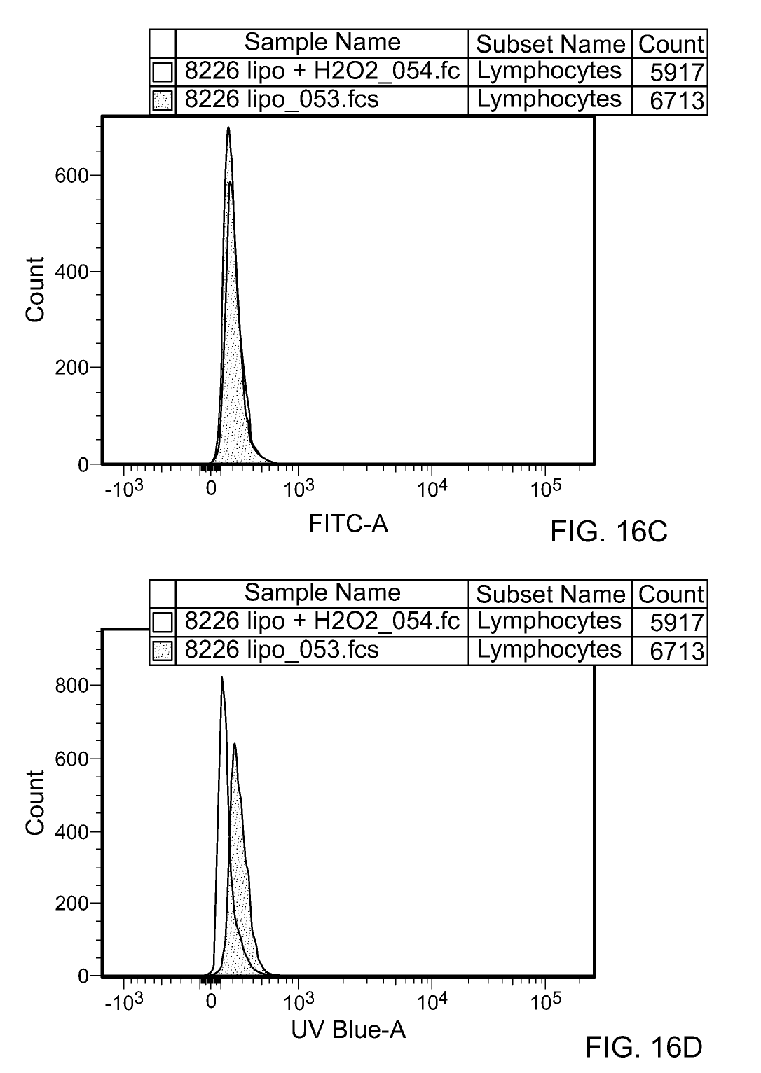

RPMI-8226 myeloma cells are very resistant to IMIDs (lenalidomide) because they exhibit high anti-oxidative capacity. External H₂O₂ treatment induced high oxidation of FADH₂ (FIG. 16A) and NAD(P)H (FIG. 16B). To decrease this anti-oxidative capacity, myeloma cells were treated with lipoic acid or lopoamide. This treatment decreased myeloma cells anti-oxidative capacity (FIGS. 16C and 16D).

These results demonstrate that lipoic acid or lopoamide can be used to induce high oxidized states of FAD and NAD(P) and thereby sensitize cells to anti-cancer drugs (e.g., cancer treatments using IMIDs such as lenalidomide).

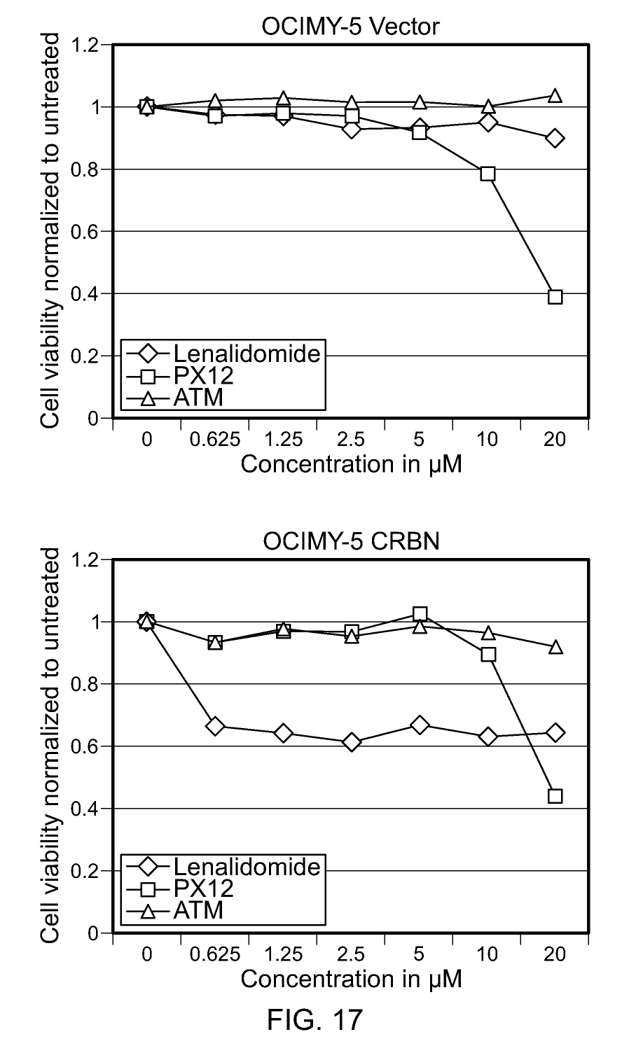

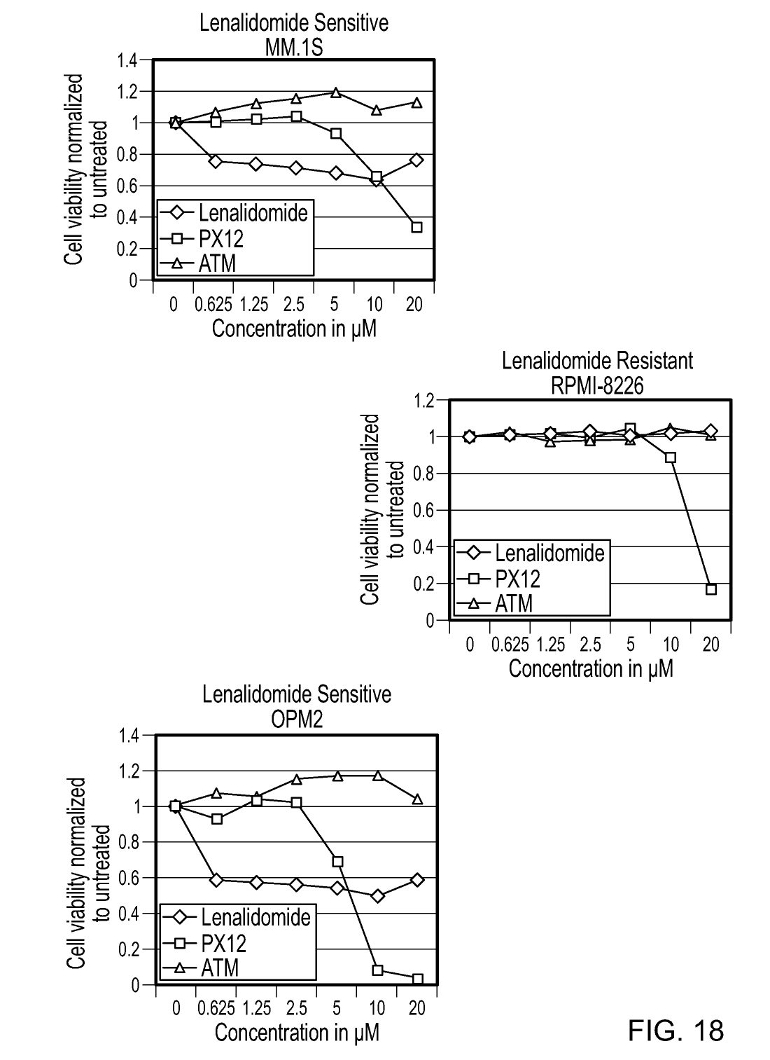

OCIMY5-Vector and OCIMY-CRBN cells were treated with lenalidomide, a thioredoxin reductase inhibitor (sodium aurothiomalate; ATM), or a thioredoxin inhibitor (PX12) for 72 hours, and cell viability was assessed using an MTT assay. ATM did not inhibit cell proliferation in both cell lines after 3 days of drug treatment (FIG. 17). Lenalidomide inhibited cell proliferation in CRBN positive cells, but not in CRBN deficient cells (FIG. 17). The thioredoxin inhibitor, PX12, inhibited cell proliferation in both cell lines, and its activity was irrespective of CRBN level (FIG. 17). Similar results were obtained with other cell lines (FIG. 18).

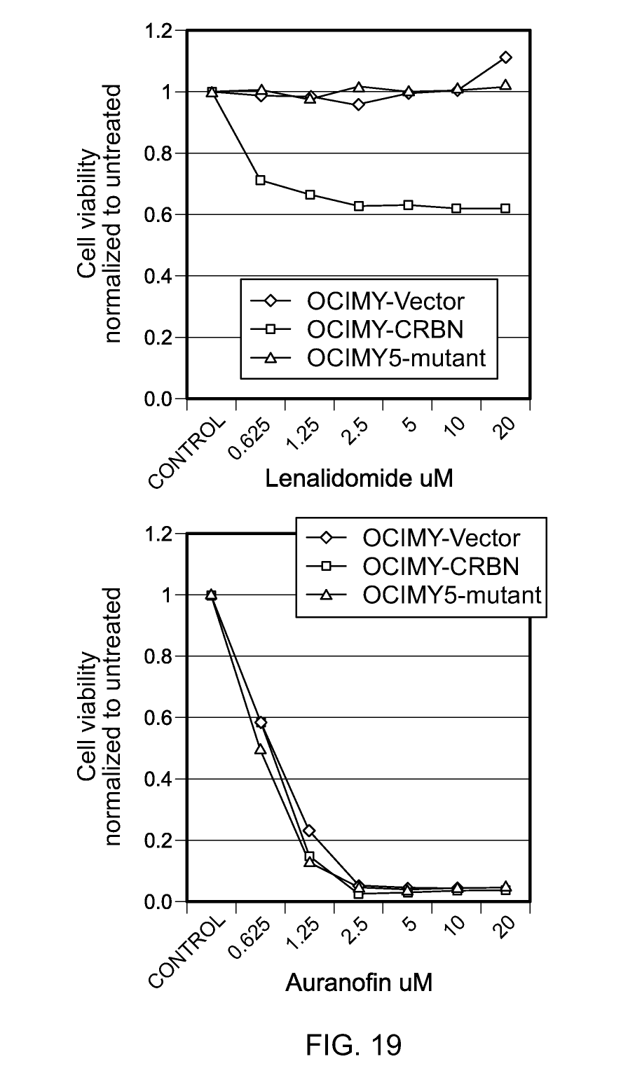

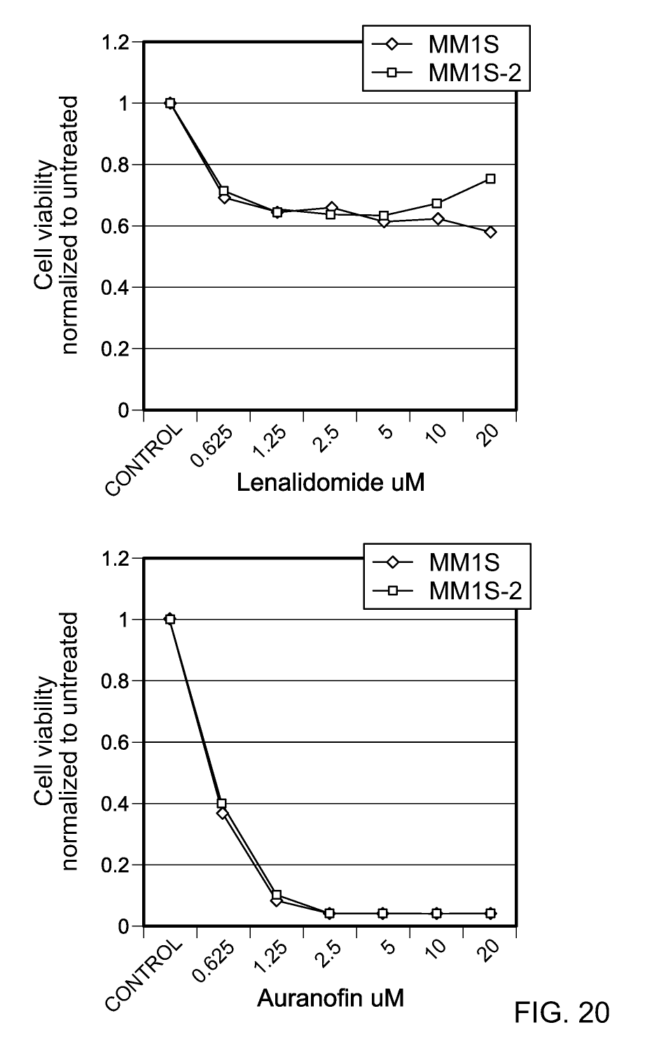

Another drug, auranofin, which inhibits thioredoxin reductase, was effective at accumulating intracellular H₂O₂ in myeloma cells and reducing cancer cell viability (FIGS. 19 and 20). Auranofin inhibited cancer cell proliferation irrespective of CRBN level and was more potent than lenalidomide in inhibiting thioredoxin reductase and cancer cell proliferation (FIG. 19-20).

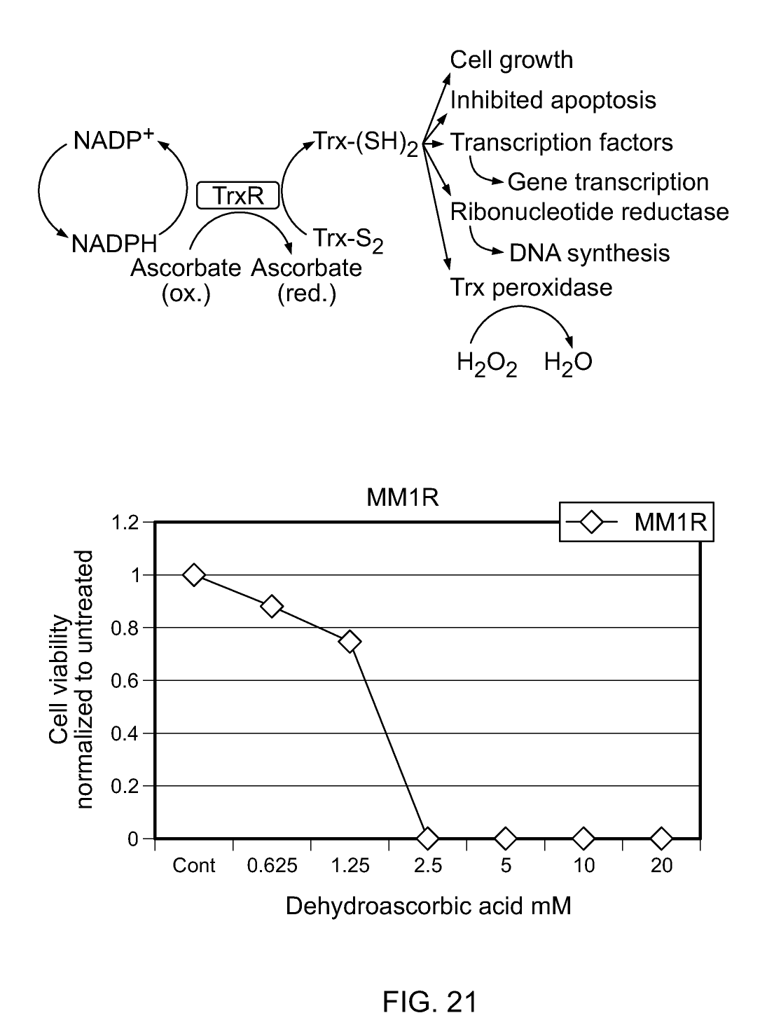

Dehydroascorbic acid (DHA) is a substrate for thioredoxin reductase and high concentrations of DHA inhibited myeloma cell proliferation (FIG. 21). These results demonstrate that a reduced form of vitamin C (e.g., DHA) at high concentrations can be used to treat multiple myeloma.

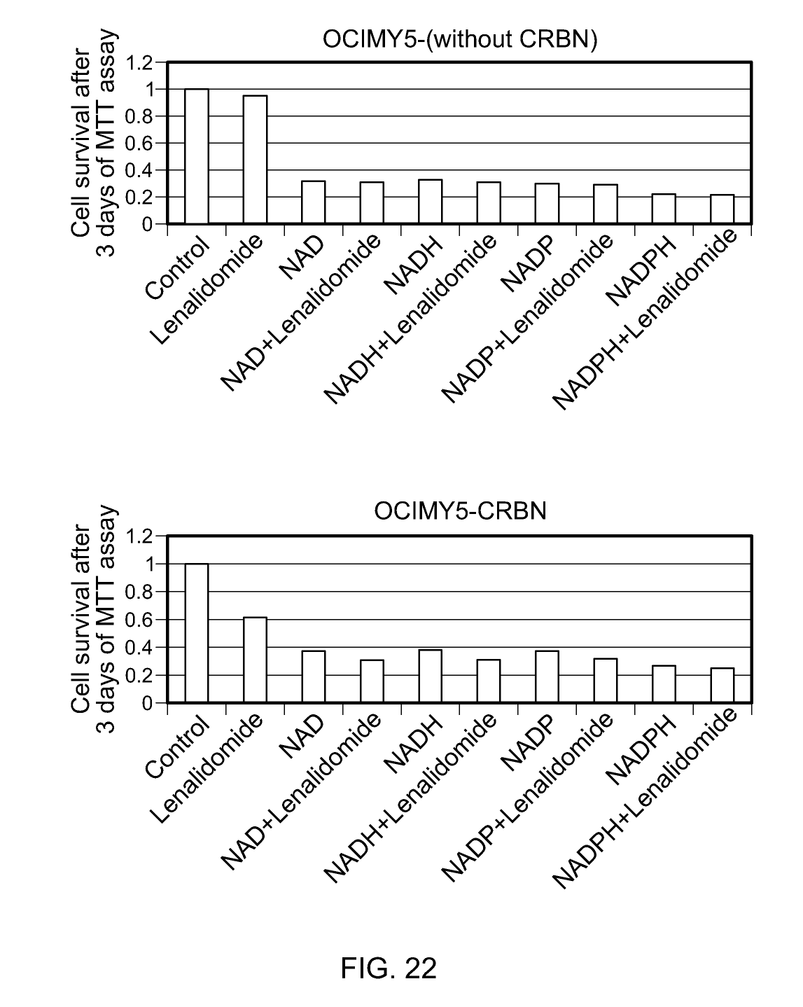

Myeloma cell line harboring CRBN or without CRBN were treated with NAD, NADH, NADP, or NADPH alone or NAD, NADH, NADP, or NADPH in combination with lenalidomide and analyzed for cell survival. Treatment with NAD, NADH, NADP, or NADPH cofactors alone or in combination with lenalidomide induced cell death in myeloma cells (FIG. 22). These results demonstrate that cancers (e.g., multiple myeloma) can be treated with NAD, NADH, NADP, and NADPH alone or in combinations with lenalidomide.

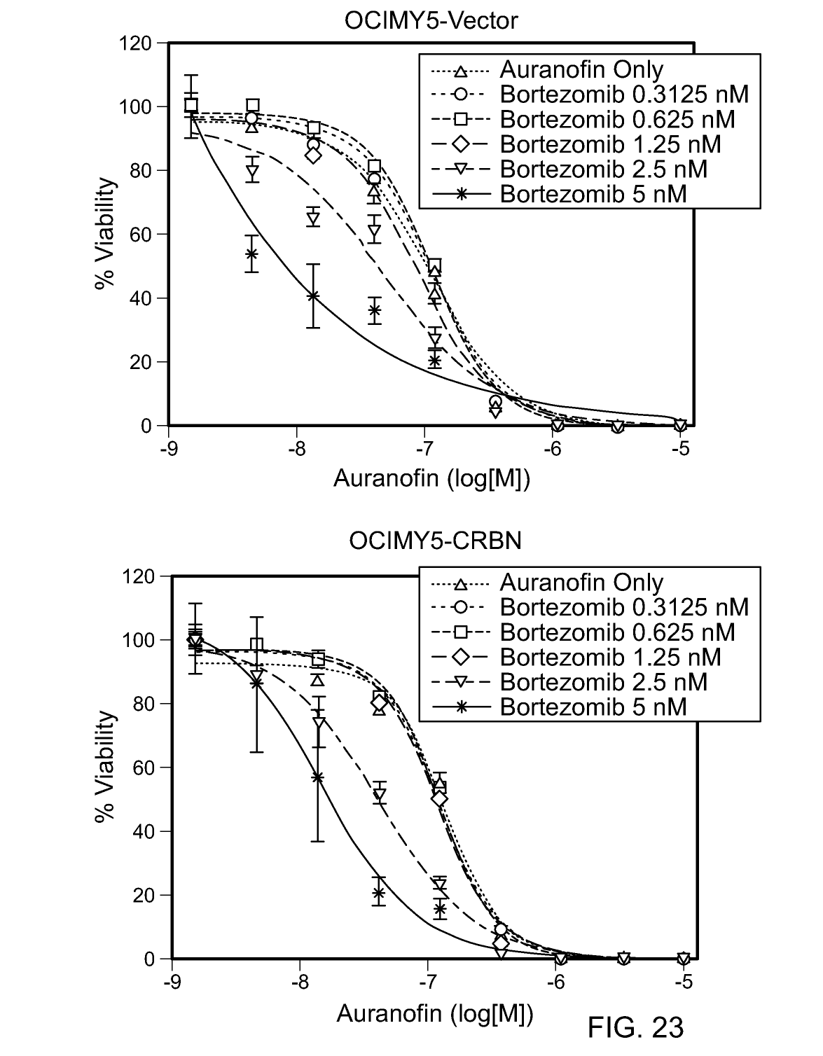

In addition, bortezomib (a proteasome inhibitors) worked in synergy with auranofin (FIG. 23). These results demonstrate that thioredoxin reductase inhibitors such as auranofin can be used in combination with a proteasome inhibitor such as bortezomib to treat cancer as described herein.

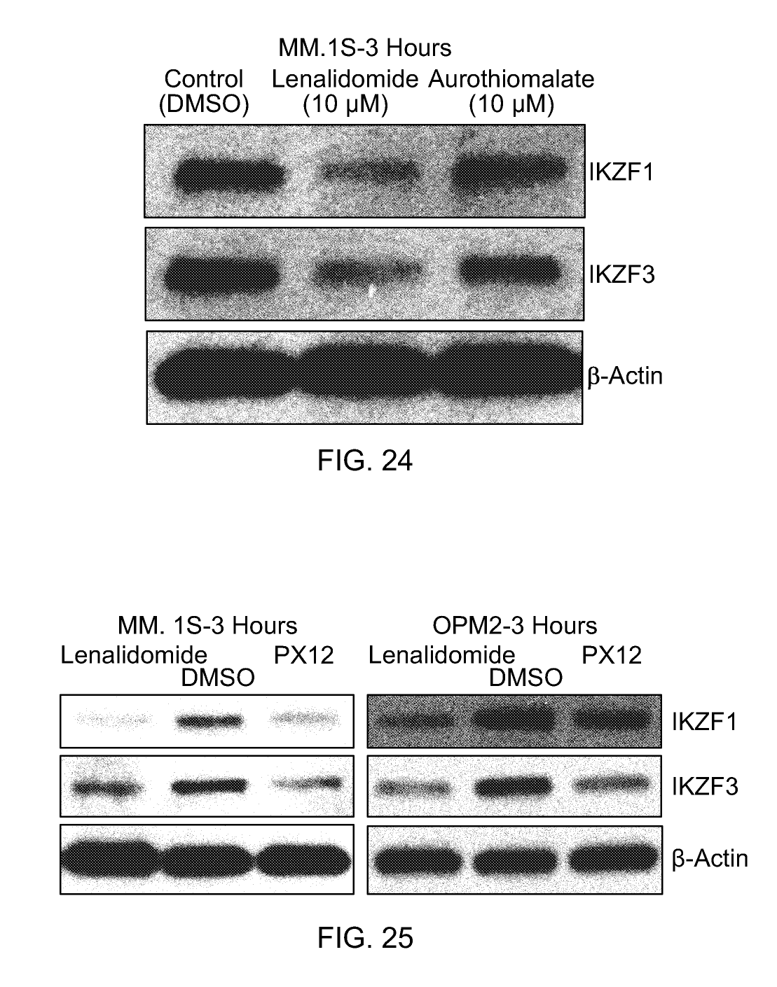

The following was performed to determine whether inhibition of thioredoxin reductase itself with aurothiomalate (FIG. 24) or with thioredoxin, a major substrate of thioredoxin reductase for maintaining intracellular H₂O₂ homeostasis, could induce specific degradation of IKZF1 and IKZF3 in MM cells. MM.1S and OPM2 cells were treated with the thioredoxin inhibitor PX12. Like lenalidomide, thioredoxin inhibition with PX12 also induced IKZF1 and IKZF3 degradation within 3 hours of treatment (FIG. 25). These results demonstrate that lenalidomide-mediated inhibition of thioredoxin reductase and subsequent thioredoxin inactivation results in elevated intracellular H₂O₂ over time, initiating the oxidization cascade that degrades IKZF1 and IKZF3 in MM.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-20. (canceled)

21. A method for treating cancer, wherein said method comprises:
(a) obtaining cancer cells from a mammal having cancer;
(b) contacting said cancer cells in a solution with an agent and exogenous H₂O₂;
(c) detecting an absence of O₂ formation in said solution; and
(d) administering said agent to said mammal under conditions wherein the number of cancer cells within said mammal is reduced.

22. The method of claim 21, wherein said mammal is a human.

23. The method of claim 21, wherein said agent is an immunomodulatory drug (IMID).

24. (canceled)

25. The method of claim 21, wherein the exogenous H₂O₂ is provided in an amount from about 20 μM to about 150 μM.

26. (canceled)

27. The method of claim 21, wherein the solution is phosphate buffered saline (PBS).

28. (canceled)

29. The method of claim 21, wherein said detecting the absence of O₂ formation in said solution comprises (i) detecting the absence of bubble formation in said solution, (ii) detecting the absence of an increase in autofluorescence of FAD, or (iii) detecting the absence of a decrease in autofluorescence of NAD(P)H.

30. The method of claim 29, wherein said detecting step comprises visually detecting the absence of formation of said bubbles.

31. A method for treating cancer, wherein said method comprises:
(a) obtaining cancer cells from a mammal having cancer;
(b) contacting at least a portion of said cancer cells in a first solution with a first agent and exogenous H₂O₂;
(c) detecting the presence of O₂ formation in said first solution;
(d) contacting at least a portion of said cancer cells in a second solution with a second agent and exogenous H₂O₂;
(e) detecting the absence of O₂ formation in said second solution; and
(f) administering said second agent to said mammal under conditions wherein the number of cancer cells within said mammal is reduced.

32. The method of claim 31, wherein said mammal is a human.

33. The method of claim 31, wherein said first agent is an immunomodulatory drug (IMID).

34. The method of claim 33, wherein said second agent is an immunomodulatory drug (IMID).

35.-37. (canceled)

38. The method of claim 31, wherein the first solution is phosphate buffered saline (PBS), and wherein the second solution is PBS.

39. (canceled)

40. The method of claim 31, wherein said detecting the presence of O₂ formation in said first solution comprises (i) detecting the presence of bubble formation in said first solution, (ii) detecting an increase in autofluorescence of FAD in said first solution, or (iii) detecting a decrease in autofluorescence of NAD(P)H in said first solution.

41. The method of claim 31, wherein said detecting the absence of O₂ formation in said second solution comprises (i) detecting the absence of bubble formation in said second solution, (ii) detecting the absence of an increase in autofluorescence of FAD in said second solution, or (iii) detecting the absence of a decrease in autofluorescence of NAD(P)H in said second solution.

42. (canceled)

43. A method for treating cancer, wherein said method comprises:
(a) obtaining cancer cells from a mammal having cancer;
(b) placing a portion of said cancers into a plurality of different containers in solution;
(c) adding a different test agent to each of said plurality of different containers;
(d) adding exogenous H₂O₂ to each of said plurality of different containers;
(e) detecting the level of O₂ formation in the solution of each of said plurality of different containers;
(f) selecting the test agent present in one of said plurality of different containers that resulted in minimal O₂ formation as compared to the level observed in at least one other of said plurality of different containers, thereby identifying said selected test agent as a treatment agent for said mammal; and
(g) administering said treatment agent to said mammal under conditions wherein the number of cancer cells within said mammal is reduced.

44. The method of claim 43, wherein said mammal is a human.

45. The method of claim 43, wherein at least one of said test agents is an immunomodulatory drug (IMID).

46-48. (canceled)

49. The method of claim 43, wherein the solution is phosphate buffered saline (PBS).

50. The method of claim 43, wherein said detecting the level of O₂ formation in the solution of each of said plurality of different containers comprises (i) detecting the level of bubble formation in the solution of each of said plurality of different containers, (ii) detecting the level of autofluorescence of FAD in the solution of each of said plurality of different containers, or (iii) detecting the level of autofluorescence of NAD(P)H in the solution of each of said plurality of different containers.

51. The method of claim 50, wherein said detecting step comprises visually detecting the level of formation of said bubbles.


SEQUENCE LISTING (6 sequences)

SEQ ID NO: 1 - DNA, 16 bp, Homo sapiens
gagcctdacgcctgag

SEQ ID NO: 2 - DNA, 22 bp, Homo sapiens
attgagggtttattgagtgcag

SEQ ID NO: 3 - DNA, 21 bp, Homo sapiens
gggtccaagttgtccagaatg

SEQ ID NO: 4 - DNA, 21 bp, Homo sapiens
gggtccaagttgtccagaatg

SEQ ID NO: 5 - DNA, 23 bp, Homo sapiens
taaagacctctatgccaacacag

SEQ ID NO: 6 - DNA, 24 bp, Homo sapiens
cacgatggaggggccggactcatc